(19) United States
(12) Patent Application Publication
(10) Pub. No.: US 2024/0182851 A1
(43) Pub. Date: Jun. 6, 2024

(54) REPROGRAMMING OF CELLS AND USES THEREOF

(71) Applicant: SQZ Biotechnologies Company, Watertown, MA (US)

(72) Inventors: Chun-I WU, Watertown, MA (US); Devin BRIDGEN, Watertown, MA (US); Jonathan B. GILBERT, Watertown, MA (US); Abdulkadir OZKAN, Watertown, MA (US); Marija TADIN-STRAPPS, Watertown, MA (US)

(73) Assignee: SQZ Biotechnologies Company, Watertown, MA (US)

(21) Appl. No.: 18/553,292

(22) PCT Filed: Apr. 4, 2022
(86) PCT No.: PCT/US2022/023356

§ 371 Date: Sep. 29, 2023

Related U.S. Application Data
(60) Provisional application No. 63/240,739, filed on Sep. 3, 2021.
(60) Provisional application No. 63/170,405, filed on Apr. 2, 2021.

(51) Int. Cl. C12N 5/0793; A61K 35/30; C12N 9/10; C12N 15/113; C12N 15/87

(52) U.S. Cl. CPC C12N 5/0619; A61K 35/30; C12N 9/1029; C12N 15/113; C12N 15/87; C12N 2310/141; C12N 2501/602; C12N 2501/999; C12N 2506/11; C12N 2506/45

(57) ABSTRACT

The present disclosure provides methods for reprogramming a cell, wherein the method comprises passing a cell suspension comprising the cell and a reprogramming factor through a constriction, wherein the constriction deforms the cell, thereby causing a perturbation of the cell such that the reprogramming factor enters the cell.

### CROSS-REFERENCE TO RELATED APPLICATION

This PCT application claims the priority benefit of U.S. Provisional Application No. 63/170,405, filed on Apr. 2, 2021; and 63/240,739, filed on Sep. 3, 2021; each of which is herein incorporated by reference in its entirety.

### FIELD OF DISCLOSURE

The present disclosure relates generally to methods of reprogramming cells to differentiate into different types of cells (e.g., neurons) through the use of one or more constrictions.

### BACKGROUND OF DISCLOSURE

Regenerative medicine based on cell replacement is on the cusp of major health impacts. However, significant challenges remain, particularly in manufacturing consistent, efficient, and cost-effective functional engraftable cells. While induced pluripotent stem cell (iPSC) differentiation and somatic cell transdifferentiation (i.e., direct reprogramming) are capable of producing various cells of interest, current methods are often cumbersome and inefficient. For instance, iPSC differentiation methods currently available can typically take up to several weeks and in some instances, even months to get to the desired terminal cell types. Furthermore, the resulting cell product is inevitably a heterogeneous population of cells with the desired cells present in wide range of frequency. Similarly, with somatic cell transdifferentiation, reprogramming by lentiviral vectors has been useful, but this method presents the risk of insertional mutagenesis. And, with methods such as electroporation and lipofection, they can negatively affect cell health and recovery of sensitive primary cells, as well as causing other cytotoxicity issues. Therefore, there remains a need for more efficient and cost-effective approaches to delivering differentiation or reprogramming factors to generate cells that can be used for various therapeutic applications, such as in regenerative medicine.

### SUMMARY OF DISCLOSURE

Provided herein is a method of producing a neuron, comprising: passing a cell suspension, which comprises a population of cells, through a constriction under one or more parameters, wherein passing the cell suspension through the constriction under the one or more parameters deforms one or more cells of the population of cells, and thereby, causing a perturbation in the cell membrane of the one or more cells, and wherein the perturbation allows a reprogramming factor to enter the one or more cells, and thereby, reprograms the one or more cells into a neuron.

In some aspects, the method further comprises contacting the population of cells with the reprogramming factor prior to passing of the cell suspension through the constriction. In some aspects, the method comprises contacting the population of cells with the reprogramming factor during the passing of the cell suspension through the constriction. In some aspects, the population of cells are contacted with the reprogramming factor during the passing of the cell suspension through the constriction. In some aspects, the method comprises contacting the population of cells with the reprogramming factor after the passing of the cell suspension through the constriction. In some aspects, the population of cells are contacted with the reprogramming factor after the passing of the cell suspension through the constriction.

In some aspects, a reprogramming factor that can be used with the methods disclosed herein comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a lncRNA, a tRNA, a shRNA, a self-amplifying mRNA (saRNA), or combinations thereof. In some aspects, the RNA is a mRNA. In some aspects, the RNA is a siRNA. In some aspects, the RNA is shRNA. In some aspects, the reprogramming factor is a small molecule, such as an impermeable small molecule.

In some aspects, the reprogramming factor comprises a transcription factor, wherein the transcription factor is capable of reprogramming the one or more cells into a neuron. In some aspects, the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor related 1 protein (Nurr1), LIM Homeobox Transcription Factor 1 Alpha (Lmx1a), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124 (mir124), or combinations thereof.

In some aspects, the reprogramming factor inhibits the expression and/or activity of p53. In some aspects, the reprogramming factor comprises a shRNA targeting p53. In some aspects, the reprogramming factor comprises a miRNA. In some aspects, the miRNA comprises miR-124.

Also provided herein is a method of inducing the reprogramming of a cell comprising: passing a cell suspension, which comprises a population of cells, through a constriction under one or more parameters, wherein passing the cell suspension through the constriction under the one or more parameters deforms one or more cells of the population of cells, and thereby, causing a perturbation in the cell membrane of the one or more cells, and wherein the perturbation allows a reprogramming factor to enter the one or more cells, and thereby, induce the reprogramming of the one or more cells.

In some aspects, the method of inducing the reprogramming of a cell further comprises contacting the population of cells with the reprogramming factor prior to passing of the cell suspension through the constriction. In some aspects, the method comprises contacting the population of cells with the reprogramming factor during the passing of the cell suspension through the constriction. In some aspects, the population of cells are contacted with the reprogramming factor during the passing of the cell suspension through the constriction. In some aspects, the method comprises contacting the population of cells with the reprogramming factor after the passing of the cell suspension through the constriction. In some aspects, the population of cells are contacted with the reprogramming factor after the passing of the cell suspension through the constriction.

In some aspects, a reprogramming factor that can be used with a method of inducing the reprogramming of a cell is capable of reprogramming the one or more cells to a neuron.

In some aspects, the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or any combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a microRNA (miRNA), a lncRNA, a tRNA, a shRNA, a self-amplifying mRNA (saRNA), or any combinations thereof. In some aspects, the RNA is a mRNA. In some aspects, the RNA is a siRNA. In some aspects, the RNA is a shRNA. In some aspects, the reprogramming factor is a small molecule, such as an impermeable small molecule.

In some aspects, the reprogramming factor comprises a transcription factor, wherein the transcription factor is capable of reprogramming the one or more cells into a neuron. In some aspects, the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor related 1 protein (Nurr1), LIM Homeobox Transcription Factor 1 Alpha (Lmx1a), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124 (mir124), or any combinations thereof.

In some aspects, the reprogramming factor inhibits the expression and/or activity of p53. In some aspects, the reprogramming factor comprises a shRNA targeting p53. In some aspects, the reprogramming factor comprises a miRNA. In some aspects, the miRNA comprises miR-124. In some aspects, the one or more cells are differentiated into a neuron.

In some aspects, the present disclosure further provides a method of increasing the delivery of a reprogramming factor into a cell, comprising modulating one or more parameters under which a cell suspension, comprising a population of cells, is passed through a constriction, wherein the population of cells are in contact with the reprogramming factor, and wherein the one or more parameters increase the delivery of the reprogramming factor into one or more cells of the population of cells compared to a reference parameter.

In some aspects, the method disclosed herein comprises contacting the population of cells with the reprogramming factor prior to passing of the cell suspension through the constriction. In some aspects, the method comprises contacting the population of cells with the reprogramming factor during the passing of the cell suspension through the constriction. In some aspects, the population of cells are contacted with the reprogramming factor during the passing of the cell suspension through the constriction. In some aspects, the method comprises contacting the population of cells with the reprogramming factor after the passing of the cell suspension through the constriction. In some aspects, the population of cells are contacted with the reprogramming factor after the passing of the cell suspension through the constriction.

In some aspects, with a method of increasing the delivery of a reprogramming factor into a cell provided herein, the delivery of the reprogramming factor into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the reprogramming factor into a corresponding cell using the reference parameter.

In any of the methods described herein, the one or more cells of the population of cells comprises stem cells, somatic cells, or both. In some aspects, the stem cells are induced pluripotent stem cells (iPSCs), embryonic stem cells, tissue-specific stem cells, mesenchymal stem cells, or combinations thereof. In some aspects, the stem cells are iPSCs. In some aspects, the somatic cells comprise blood cells. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

For any of the methods described herein, in some aspects, the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload; or combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, a constriction that is used with any of the methods provided herein is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells.

In any of the methods provided herein, in some aspects, the length of the constriction is up to 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

In any of the methods provided herein, in some aspects, the population of cells are contacted with multiple reprogramming factors, such that at least two or more of the multiple reprogramming factors are cable of entering the one or more cells after the perturbation of the cell membrane. In some aspects, the multiple reprogramming factors comprise at least about 2, at least about 3, at least about 4, at least about 5, at least about 6, at least about 7, at least about 8, at least about 9, or at least about 10 or more reprogramming factors. In some aspects, the multiple reprogramming factors are delivered into the cells concurrently. In some aspects, one or more of the reprogramming factors are delivered into the cells sequentially.

In any of the methods provided herein, in some aspects, the method comprises passing the cell suspension through a plurality of constrictions. In some aspects, the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, each constriction of the plurality of constrictions are the same. In some aspects, one or more of the constrictions of the plurality of constrictions are different. In some aspects, the one or more of the constrictions differ in their length, depth, width, or combinations thereof. In some aspects, the one or more of the constrictions differ in their length, depth, width, or combinations thereof. In some aspects, one or more of the plurality of constrictions is associated with a different reprogramming factor. In some aspects, the plurality of constrictions comprise a first constriction associated with a first reprogramming factor and a second constriction associated with a second reprogramming factor, wherein the cell suspension passes through the first constriction such that the first reprogramming factor is delivered to one or more cells of the plurality of cells, and then the cell suspension passes through the second constriction such that the second reprogramming factor is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In any of the methods provided herein, in some aspects, the method further comprises contacting the plurality of cells with an additional compound.

In some aspects, the additional compound comprises a nucleic acid, a polypeptide, a lipid, a carbohydrate, a small molecule, a metal-containing compound, an antibody, a transcription factor, a nanoparticle, a liposome, a fluorescently tagged molecule, or any combinations thereof. In some aspects, the additional compound is a nucleic acid encoding an enzyme that provides resistance to an antibiotic. In some aspects, the contacting of the additional compound with the plurality of cells occurs concurrently with the reprogramming factor. In some aspects, the contacting of the additional compound with the plurality of cells occurs prior to or after the contacting of the plurality of cells with the reprogramming factor.

In some aspects, a method provided herein further comprises collecting the cell suspension that passed through the constriction and treating the cell suspension with the antibiotic. In some aspects, the enzyme (e.g., encoded by the additional compound) comprises puromycin-N-acetyltransferase, and the antibiotic is puromycin. In some aspects, after the treatment with the antibiotic, the proportion of neurons present in the cell suspension is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold.

Also provided herein is a composition comprising a population of differentiated cells produced by any of the methods provided herein.

In some aspects, the present disclosure additionally provides a composition comprising a population of cells and a reprogramming factor under one or more parameters resulting in deformation of one or more cells of the population of cells, wherein the one or more cells comprise a perturbation in the cell membrane sufficient to allow the reprogramming factor to enter the one or more cells.

In some aspects, the one or more cells of the population of cells comprises stem cells, somatic cells, or both. In some aspects, the stem cells are induced pluripotent stem cells (iPSCs), embryonic stem cells, tissue-specific stem cells, mesenchymal stem cells, or combinations thereof. In some aspects, the stem cell is iPSCs. In some aspects, the somatic cells comprise blood cells. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

In any of the compositions provided herein, in some aspects, the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a miRNA, a lncRNA, a tRNA, a shRNA, a self-amplifying mRNA (saRNA), or combinations thereof. In some aspects, the RNA is a mRNA.

In any of the compositions provided herein, in some aspects, the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a payload concentration; a viscosity, osmolarity, salt concentration, serum content, and/or pH of the cell suspension; time in the constriction; shear rate in the constriction; type of payload; or any combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells.

In any of the compositions provided herein, in some aspects, the length of the constriction is up to 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

Also provided herein is a cell comprising a perturbation in the cell membrane due to one or more parameters which deform the cell thereby causing the perturbation in the cell membrane of the cell such that a reprogramming factor can enter the cell. Also provided herein is a cell comprising a reprogramming factor, wherein the reprogramming factor entered the cell through a perturbation in the cell membrane due to one or more parameters which deformed the cell thereby causing the perturbation in the cell membrane of the cell such that the reprogramming factor entered the cell.

In some aspects, the cell comprises stem cells, somatic cells, or both. In some aspects, the stem cells are induced pluripotent stem cells (iPSCs), embryonic stem cells, tissue-specific stem cells, mesenchymal stem cells, or combinations thereof. In some aspects, the stem cell is iPSCs. In some aspects, the somatic cells comprise blood cells. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

In any of the cells provided herein, in some aspects, the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, a mRNA, a miRNA, a lncRNA, a tRNA, a shRNA, a self-amplifying mRNA (saRNA), or combinations thereof. In some aspects, the RNA is a mRNA. In some aspects, the RNA is a siRNA. In some aspects, the RNA is a shRNA. In some aspects, the reprogramming factor is a small molecule, such as an impermeable small molecule.

In some aspects, the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a payload concentration; a viscosity, osmolarity, salt concentration, serum content, and/or pH of the cell suspension; time in the constriction; shear rate in the constriction; type of payload; or any combinations thereof.

In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In any of the cells provided herein, in some aspects, the constriction is within a microfluidic chip. In some aspects, the diameter of the constriction is about 20%, about 30%, about 40%, about 50%, about 60%, about 70%, about 80%, about 90%, or about 99% of the diameter of the one or more cells of the population of cells.

In some aspects, the length of the constriction is up to 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

In some aspects, the present disclosure provides a method of treating a neurological disorder in a subject in need thereof, comprising administering to the subject a plurality of neurons, wherein the neurons are produced by any of the methods provided. Also provided herein is a method of treating a neurological disorder in a subject in need thereof, comprising administering to the subject any of the composition or cells described herein. In some aspects, the neurological disorder comprises a Parkinson's disease.

### BRIEF DESCRIPTION OF FIGURES

FIG. 1A-FIG. 1E present graphical representations of the viability, percentage payload delivery, and fluorescent MFI of iPSCs after squeeze delivery with either 3 kDa dextran-cascade blue and/or 70 kDa dextran-fluorescein under different pressures in either Opti-MEM or StemFlex basal medium, as described in Example 1. FIG. 1A shows cell viability after squeeze delivery of 3 kDa dextran-cascade blue and 70 kDa dextran-fluorescein under the different pressures. FIGS. 1B and 1C show percentage payload delivery after squeeze delivery of 3 kDa dextran-cascade blue 70 kDa dextran-fluorescein, respectively, under the different pressures. FIGS. 1D and 1E show fluorescent MFI after squeeze delivery of 3 kDa dextran-cascade blue and 70 kDa dextran-fluorescein, respectively, under the different pressures. The x-axis provides a description of the constriction and pressure used. “10-6-70” refers to the dimensions of the constriction used (length of 10 μm, width of 6 μm, and depth of 70 μm). The pressure were as follows: 30 psi, 45 psi, 60 psi, 75 psi, or 90 psi. “Opti” refers to Opti-MEM. “Stem” refers to StemFlex basal medium.

FIG. 2A-FIG. 2C present graphical representations of the viability (FIG. 2A), percentage delivery (FIG. 2B), and fluorescent MFI (FIG. 2C) of iPSCs 24 hours post-squeeze delivery with EGFP mRNA using various different cell concentrations, as described in Example 2. The X-axis provides the cell concentrations used.

FIG. 3A-FIG. 3C present graphical representations of the viability (FIG. 3A), percentage delivery (FIG. 3B), and fluorescent MFI (FIG. 3C) of iPSCs after squeeze delivery with Cyanine 5 EGFP mRNA using various different pressures, as described in Example 3. The different pressures, which are shown along the x-axis, were as follows: 45 psi, 60 psi, or 75 psi.

FIG. 4 presents a graphical representation of the fold change in expression of five different iPSC pluripotent marker genes (Oct4, Nanog, Sox2, MYC, and TERT) in squeeze processed iPSCs after 24 hours squeeze treatment, as described in Example 4. The expression of the different marker genes in the squeeze processed genes is shown relative to the corresponding expression in the no squeeze processed control cells. The gene expression levels were analyzed by RT-qPCR 24 hours after squeeze delivery.

FIG. 5 presents a graphical representation of the percentage of cells expressing iPSC pluripotent protein markers (Oct4, Sox2, SSEA4) 24 hours after squeeze treatment as compared to iPSCs not treated with a squeeze treatment, as described in Example 4. The top and bottom left panels represent controls that were not subjected to squeeze treatment, and the top and bottom right panels represent iPSCs that were squeeze treated. The X-axis of the top and bottom panels represents the percentage of cells expressing SSEA4, the Y-axis of the top panels represents the percentage of cells expressing SOX2, and the Y-axis of the bottom panels represents the percentage of cells expressing Oct4. The expression levels were measured by flow cytometry 24 hours after squeeze treatment.

FIG. 6 presents a graphical representation comparing the Ct values of 32 housekeeping genes between no squeeze treatment and squeeze treated iPSCs, as described in Example 4. The white bars represent iPSCs that were not squeeze treated and the solid black bars represent squeeze-treated iPSCs. Gene expression was measured by RT-qPCR 24 hours after squeeze treatment.

FIG. 7 presents Western blot analysis of iPSCs that were squeeze treated with Ngn2 mRNAs, as described in Example 5. Each gel lane was loaded with iPSCs squeezed with the mRNA as indicated in the gel image. The different mRNAs included: (1) SA-Ngn2 mRNA (comprises a serine to alanine mutation) (“mRNA1”); (2) SA-co-Ngn2 mRNA (codon optimized) (“mRNA2”); (3) Sa-co-m6AG-Ngn2 mRNA (lower decapping rate) (“mRNA3”); (4) SA-co-KOCO-Ngn2 mRNA (comprises 10 lysine to arginine mutations and 6 cysteine to alanine mutations to block ubiquitination) (“mRNA4”); and (5) SA-co-Kozak-Ngn2 mRNA (second amino acid is mutated to alanine to fit Kozak consensus sequence) (“mRNA5”). Staining was performed for Ngn2 and for GAPDH (control).

FIG. 8A-FIG. 8B present graphical representations of the kinetics of Ngn2 downstream target genes NeuroD1 (FIG. 8A) and NeuroD4 (FIG. 8B) after squeeze delivery of different Ngn2 mRNAs, as described in Example 6. The different mRNAs tested included: (1) SA-co-Ngn2 mRNA (codon optimized) (“mRNA1”); (2) Sa-co-m6AG-Ngn2 mRNA (lower decapping rate) (“mRNA2”); (3) SA-co-m6AG-DBG-Ngn2 mRNA (DBG: double human β-globin 3′UTR) (“mRNA3”); (4) SA-co-Kozak-Ngn2 mRNA second amino acid is mutated to alanine to fit Kozak consensus sequence) (“mRNA4”); and (5) SA-co-Kozak-AES-Ngn2 mRNA (AES: AES-mtRNR1 3′UTR, AES: Amino-terminal enhancer of split, mtRNR1: Mitochondrially encoded 12S rRNA) (“mRNA5”). Total RNA was isolated for NeuroD1 and NeuroD4 and expression analyzed with RT-qPCR at 12, 24, 36, and 48 hours post squeeze delivery (X-axis).

FIG. 9A-FIG. 9D present phase contrast images of iPSCs that were squeeze delivered with Ngn2, PAC, or Ngn2 with PAC, and treated with 12 hours of puromycin selection, as described in Example 7. FIG. 9A presents phase contrast images of iPSCs that were squeeze treated with no material and either not treated with puromycin (top panel) or treated with puromycin (bottom panel). FIG. 9B presents phase contrast images of iPSCs that were squeeze treated with Ngn2 (TF) mRNA and either not treated with puromycin (top panel) or treated with puromycin (bottom panel). FIG. 9C presents phase contrast images of iPSCs that were squeeze treated with PAC mRNA and either not treated with puromycin (top panel) or treated with puromycin (bottom panel). FIG. 9D presents phase contrast images of iPSCs that were squeeze treated with Ngn2 (TF) mRNA and PAC mRNA and treated with puromycin.

FIG. 10A-FIG. 10D present fluorescent images of iPSCs codelivered with Ngn2 and PAC mRNA and fixed at 24 hours after squeeze delivery, as described in Example 8. Cells were either positively immunostained with iPSC pluripotent marker Nanog or early neuronal marker Tuj1. FIG. 10A presents a fluorescent image of cells stained for Nanog. FIG. 10B presents a fluorescent image of cells stained for TUJ1. FIG. 10C presents a fluorescent image of cells in which the nucleus was stained. FIG. 10D presents a merged fluorescent image of FIG. 10A-FIG. 10C.

FIG. 11A-FIG. 11C present fluorescent images of iPSCs codelivered by squeeze delivery with Ngn2 and PAC mRNA, treated with puromycin for 12 hours, treated with Arac for 4 days, and then fixed at day 7 following squeeze delivery, as described in Example 7. The cells were stained with MAP2 (FIG. 11A) and TUJ1 (FIG. 11B) as indicated. FIG. 11C provides a merged overlay of images provided in FIGS. 11A and 11B.

FIG. 12A-FIG. 12D present graphical representations of the Nanog and TUJ1 expression levels of iPSCs squeeze delivered with Ngn2 and PAC mRNA using various different pressures, as described in Example 8. The expression levels were measured using flow cytometry. The different pressures tested included: 30 psi (FIG. 12A), 45 psi (FIG. 12B), 60 psi (FIG. 12C), and 75 psi (FIG. 12D).

FIG. 13A-FIG. 13E present graphical representations of Ngn2 protein level of iPSCs squeeze delivered with Ngn2_T2A_GFP or Ngn2 with GFP mRNA using various different pressures and measured by GFP immunofluorescence signal, as described in Example 9. FIG. 13A shows Ngn2 protein expression in iPSCs that were not squeeze treated. FIG. 13B shows Ngn2 protein expression in iPSCs squeeze delivered with Ngn2_T2A_GFP (bottom panel) or Ngn2 with GFP mRNA (top panel) using 45 psi. FIG. 13C shows Ngn2 protein expression in iPSCs squeeze delivered with Ngn2_T2A_GFP (bottom panel) or Ngn2 with GFP mRNA (top panel) using 45 psi. FIG. 13D shows Ngn2 protein expression in iPSCs squeeze delivered with Ngn2_T2A_GFP (bottom panel) or Ngn2 with GFP mRNA (top panel) using 60 psi. FIG. 13E shows Ngn2 protein expression in iPSCs squeeze delivered with Ngn2_T2A_GFP (bottom panel) or Ngn2 with GFP mRNA (top panel) using 75 psi.

FIG. 14 presents Western blot analysis comparing four different Atoh1 mRNA constructs in contributing to the Atoh1 expression at 6 and 20 hours after squeeze delivery into iPSCs, as described in Example 10. Total proteins were harvested at 6 and 20 hours for Atoh1 immunoblotting.

FIG. 15A-FIG. 15D present graphical representations of the expression levels of Atoh1 downstream target genes NeuroD1 and NeuroD4 in iPSCs squeeze delivered with Atoh1 mRNA, as described in Example 10. Total RNA was isolated at 12 and 24 hours post squeeze delivery. FIG. 15A shows NeuroD1 expression at 12 hours after squeeze delivery. FIG. 15B shows NeuroD1 expression at 24 hours after squeeze delivery. FIG. 15C shows NeuroD4 expression at 12 hours after squeeze delivery. FIG. 15D shows NeuroD4 expression at 24 hours after squeeze delivery.

FIG. 16 shows the percentage of viable PBMCs observed after first and second squeeze delivery of multiple payloads. The PBMCs were squeeze delivered either (i) a combination of four different synthetic mRNAs encoding the following neurogenic factors: BRN2, ASCL1, MYTIL, or NGN2 (“BAMN”); or (ii) a combination of eight different synthetic mRNAs encoding the following neurogenic factors: NURR1, FOXA2, LMXIA, BRN2, ASCL1, MYTIL, NGN2, or SOX2 (“NFL-BAMN-S”). PBMCs that were not squeeze processed (“no contact”) and squeeze processed with no payload (“empty squeeze”) were used as controls. Cell viability is shown for the following time points: (1) immediately after first squeeze processing (“0 hr-post-1^{st} squeeze”), (2) 24 hours after first squeeze processing (“24 hr-post-1^{st} squeeze”), (3) immediately after second squeeze processing (“0 hr-post-2^{nd} squeeze”), and (4) 24 hours after second squeeze processing (“48 hr-post-2^{nd} squeeze”).

FIG. 17A-FIG. 17B show the expression of Nurr1 and Tuj1, respectively, in PBMCs after first and second squeeze delivery of multiple payloads, as measured using qPCR analysis. The PBMCs were squeeze delivered either (i) a combination of four different synthetic mRNAs encoding the following neurogenic factors: BRN2, ASCL1, MYT1L, or NGN2 (“BAMN”); or (ii) a combination of eight different synthetic mRNAs encoding the following neurogenic factors: NURR1, FOXA2, LMX1A, BRN2, ASCL1, MYT1L, NGN2, or SOX2 (“NFL-BAMN-S”). PBMCs that were not squeeze processed (“no contact”) were used as control. Nurr1 and Tuj1 expression is shown as fold change over the expression observed in the “no contact” group.

FIG. 18A-FIG. 18F show the expression of various neuronal and dopaminergic neuron lineage markers (i.e., NeuroD1, FoxA2, Pitx3, TH, Neurog2, and Lmx1a, respectively) in iPSCs after squeeze processing. A combination of mRNAs encoding the following reprogramming factors was delivered to the iPSCs using a single squeeze processing: Ascl1, Lmx1a, Nurr1, FoxA2, and PAC (“AFLN-P”). iPSCs that underwent squeeze processing but without any payload (e.g., reprogramming factors) were used as control (“Control”). The expression of the different markers are shown as fold increase over the expression observed in the control group.

FIG. 19 provides Western blot analysis showing the expression of the following neuronal and dopaminergic neuron lineage markers in iPSCs after squeeze processing: Ascl1, Lmx1a, Nurr1, and FoxA2. A combination of mRNAs encoding the following reprogramming factors was delivered to the iPSCs using a single squeeze processing: Asc11, Lmx1a, Nurr1, and FoxA2 (“ALNF”; lanes #2 and #4). iPSCs that underwent squeeze processing but without any payload (e.g., reprogramming factors) were used as control (“Control”; lanes #1 and #3). The expression of the markers are shown as measured at two different time points, 4 hours (lanes #1 and #2) and 8 hours (lanes #3 and #4).

FIGS. 20A-20E show the expression of of different neuronal and dopaminergic neuron lineage markers (i.e., NeuroD1, FoxA2, TH, Pitx3, Lmx1a, and Nurr1, respectively) in iPSCs after the following concurrent squeeze delivery: (1) cocktail of PAC mRNA+six codon-optimized mRNAs encoding Ascl1, Lmx1a, Nurr1, FoxA2, Pitx3, and EN1 (“6TFs+PAC”; triangle); or (2) cocktail of PAC mRNA+four codon-optimized mRNAs encoding Ascl1, Lmx1a, Nurr1, and FoxA2 (“4TFs+PAC”; square). iPSCs that underwent squeeze processing but without any payload (e.g., reprogramming factors) were used as control (“Squeeze only”; circle). The expression of the different markers are shown as fold increase over the expression observed in the control group at 8 hours, 24 hours, and 48 hours post squeeze delivery.

FIGS. 21A-21C show cell viability and cell retention (i.e., cell recovery) in CD34+HSCs after squeeze delivery of (i) mRNA encoding ASCL1 only or (ii) a cocktail of mRNAs encoding BRN2, ASCL1, MYT1L, and NGN2 (BAMN). CD34+ cells that were not squeeze processed (“no contact”) and squeeze processed with no payload (“empty sqz”) were used as controls. FIG. 21A shows the percentage of viable cells at 0 hour post squeeze delivery. FIG. 21B shows the percentage of viable cells at 24 hours post squeeze delivery. FIG. 21C shows the the percentage of cells recovered after squeeze delivery.

FIGS. 22A-22D show relative mRNA expression level in CD34+HSCs after squeeze delivery of (i) mRNA encoding ASCL1 only or (ii) a cocktail of mRNAs encoding BRN2, ASCL1, MYT1L, and NGN2 (BAMN). CD34+ cells that were not squeeze processed (“no contact”) and squeeze processed with no payload (“empty sqz”) were used as controls. FIG. 22A provides a comparison of the total mRNA level. FIG. 22B provides a comparison of the level of TUJ1 mRNA. FIG. 22C provides a comparison of the level of ZBTB18 mRNA. FIG. 22D provides a comparison of the level of SOX2 mRNA. For each of the figures, the mRNA level is shown relative to the corresponding expression observed in the no contact cells at 0 hour, 4 hours, 8 hours, and 24 hours after squeeze delivery.

FIG. 23 shows the percentage of viable cells after 1^{st}, 2^{nd}, and 3rd squeeze delivery of reporter mRNAs in human PBMCs. As described in Example 16, PBMCs received three separate reporter mRNAs using three successive squeeze processing steps (at 0 hour, 24 hours, and 48 hours) (“Squeeze”; black bars). PBMCs that were not squeeze processed were used as control (“No Contact”; gray bar). Cell viability was measured at the following timepoints: (1) immediately after 1^{st} squeeze delivery (“1^{st} Squeeze 0 hr”), (2) 24 hours after 1^{st} squeeze delivery (“1^{st} Squeeze 24 hr”), (3) immediately after 2^{nd} squeeze delivery (“2^{nd} Squeeze 0 hr”), (4) 24 hours after 2^{nd} squeeze delivery (“2″d Squeeze 24 hr”), and (5) immediately after 3rd squeeze delivery (“3rd Squeeze 0 hr”).

FIG. 24 shows the percentage of cells recovered after 1^{st} (“1^{st} Squeeze”), 2^{nd} (“2^{nd} Squeeze”), and 3rd (“3rd Squeeze”) squeeze delivery of reporter mRNAs in human PBMCs. As described in Example 16, PBMCs received three separate reporter mRNAs using three successive squeeze processing steps (at 0 hour, 24 hours, and 48 hours) (“Squeeze”; black bars). PBMCs that were not squeeze processed were used as control (“No Contact”; gray bar).

FIGS. 25A-25C shows the percentage of cells expressing different reporter proteins after successive squeeze delivery of mRNA encoding the different reporter proteins. FIG. 25A shows the percentage of PBMCs expressing GFP after 1^{st} squeeze delivery of GFP-encoding reporter mRNA (EGFP). FIG. 25B shows the percentage of PBMCs expressing (i) GFP only, (ii) mCherry (RFP) only, or (iii) both GFP and mCherry (“GFP-RFP”) after 2^{nd} squeeze delivery of mCherry-encoding reporter mRNA (“EGFP>RFP”). For comparison, PBMCs that received only a single squeeze delivery of RFP is also shown (“RFP”). FIG. 25C shows the percentage of PBMCs expressing (i) GFP only, (ii) mCherry (RFP) only, (iii) Dextran (DEX) only, (iv) both GFP and RFP, (v) both GFP and Dex, (vi) both RFP and Dex, and (viii) GFP, RFP, and Dex after 3rd squeeze delivery of Dextran-encoding reporter mRNA (“EGFP>RFP>Dextran”). For comparison, PBMCs that received only a single squeeze delivery of Dextran is also shown (“Dextran”). In each of the figures, “negative” corresponds to cells that do not express the particular reporter protein. In each of the figures, “control” group refers to PBMCs that were not squeeze processed.

### DETAILED DESCRIPTION OF DISCLOSURE

The present disclosure is generally directed to methods of producing neurons. More particularly, the methods provided herein comprise passing a cell suspension comprising a population of cells through a constriction under one or more parameters, such that the cells are transiently deformed, resulting in the perturbation of the cell membrane of the cells. The cell suspension can further comprise one or more reprogramming factors, so that the perturbation of the cell membrane allows the reprogramming factors to enter the cells, and thereby, induce one or more cells of the population of cells to differentiate into neurons. Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4th ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6th ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

The term “heterogeneous” as used herein refers to something which is mixed or not uniform in structure or composition. In some aspects, the term refers to pores having varied sizes, shapes, or distributions within a given surface.

The term “homogeneous” as used herein refers to something which is consistent or uniform in structure or composition throughout. In some aspects, the term refers to pores having consistent sizes, shapes, or distribution within a given surface.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

### III. Methods of the Disclosure

In some aspects, the present disclosure relates to methods of delivering a cargo (also referred to herein as “payload”) into a cell by passing the cells through a constriction (such as those described herein). As demonstrated herein, as the cells pass through the constriction, they become transiently deformed, such that cell membrane of the cells is perturbed. The perturbations within the cell membrane can allow various payloads to enter or loaded into the cell (e.g., through diffusion). The specific process by which the cells pass through a constriction and become transiently deformed is referred to herein as “squeeze processing” or “squeezing.”

As demonstrated herein, through such methods, different aspects of a cell can be modified. In some aspects, by using a payload that is capable of reprogramming a cell (i.e., “reprogramming factor”), the squeeze processing methods provided herein can be used to produce different cells of interest. While the present disclosure generally discloses the use of reprogramming factors, it will be apparent to those skilled in the art that disclosures related to reprogramming factors can equally apply to other types of payloads. Unless indicated otherwise, the terms “payload” and “reprogramming factor” are used interchangeably.

Accordingly, in some aspects, provided herein is a method of producing a neuron, wherein the method comprises passing a cell suspension through a constriction under one or more parameters, wherein the cell suspension comprises a plurality of cells, wherein one or more cells of the plurality of cells become transiently deformed as they pass through the constriction, resulting in perturbations in the cell membrane of the one or more cells, which allow a reprogramming factor to enter the cells and reprogram the cells to become neurons. Examples of reprogramming factors that can be used with the present disclosure are provided elsewhere in the present disclosure.

In some aspects, the above method further comprises contacting the population of cells with the reprogramming factor prior to passing the cell suspension through the constriction. For instance, in some aspects, prior to passing the cell suspension through the constriction, the method comprises contacting the population of cells with a reprogramming factor to produce the cell suspension. In some aspects, a method of producing a neuron described herein comprising contacting the population of cells with the reprogramming factor as the cells pass through the constriction. In some aspects, the population of cells are first contacted with the reprogramming factor during the passing of the cell suspension through the constriction. In some aspects, the population of cells are in contact with the reprogramming factor both prior to the passing step (i.e., passing of the cell suspension through the constriction) and during the passing step. In some aspects, the method comprises contacting the population of cells with the reprogramming factor after the passing of the cell suspension through the constriction. In some aspects, the population of cells are first contacted with the reprogramming factor after the passing of the cell suspension through the constriction. In some aspects, the population of cells are in contact with the reprogramming factor prior to, during, and/or, after the passing step. As further described elsewhere in the present disclosure, when the population of cells are contacted with the reprogramming factor after the passing step, the contacting occurs after the cells have passed through the constriction, such that there are still perturbations within the cell membrane.

As used herein, the “contacting” that can occur between a cell and a payload (e.g., reprogramming factor) includes that a cell can be in contact with the payload as long as the payload is capable of entering the cell once there are perturbations within the cell membrane of the cell. To help illustrate, in some aspects, a cell and a payload are in contact if they are both present within the same cell suspension.

### III.A. Cell Suspensions

In some aspects, a cell suspension described herein comprises any suitable cells known in the art that can be modified (e.g., by introducing a reprogramming factor) using the squeeze processing methods described herein.

In some aspects, the cells are stem cells. As used herein, the term “stem cells” refer to cells having not only self-replication ability but also the ability to differentiate into other types of cells (e.g., neurons). In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof. In some aspects, the stem cells are iPSCs.

In some aspects, the cells are somatic cells. As used herein, the term “somatic cells” refer to any cell in the body that are not gametes (sperm or egg), germ cells (cells that go on to become gametes), or stem cells. Non-limiting examples of somatic cells include blood cells, bone cells, muscle cells, nerve cells, or combinations thereof. In some aspects, somatic cells useful for the present disclosure comprise blood cells. In some aspects, the blood cells are peripheral blood mononuclear cells (PBMCs). As used herein, “PBMCs” refer to any peripheral blood cells having a round nucleus. In some aspects, PBMCs comprise an immune cell. As used herein the term “immune cell” refers to any cell that plays a role in immune function. In some aspects, immune cell comprises a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the blood cells are red blood cells. In some aspects, the cell is a cancer cell. In some aspects, the cancer cell is a cancer cell line cell, such as a HeLa cell. In some aspects, the cancer cell is a tumor cell. In some aspects, the cancer cell is a circulating tumor cell (CTC). In some aspects, the cell is a fibroblast cell, such as a primary fibroblast or newborn human foreskin fibroblast (Nuff cell). In some aspects, the cell is an immortalized cell line cell, such as a HEK293 cell or a CHO cell. In some aspects, the cell is a skin cell. In some aspects, the cell is a reproductive cell such as an oocyte, ovum, or zygote. In some aspects, the cell is a cluster of cells, such as an embryo, given that the cluster of cells is not disrupted when passing through the pore.

In some aspects, the cell suspension useful for the present disclosure comprises a mixed or purified population of cells. In some aspects, the cell suspension is a mixed cell population, such as whole blood, lymph, PBMCs, or combinations thereof. In some aspects, the cell suspension is a purified cell population. In some aspects, the cell is a primary cell or a cell line cell.

As demonstrated herein, the delivery of a payload (e.g., reprogramming factor) into a cell can be regulated through one or more parameters of the process in which a cell suspension is passed through a constriction. In some aspects, the specific characteristics of the cell suspension can impact the delivery of a payload into a cell. Such characteristics include, but are not limited to, osmolarity, salt concentration, serum content, cell concentration, pH, temperature or combinations thereof.

In some aspects, the cell suspension comprises a homogeneous population of cells. In some aspects, the cell suspension comprises a heterogeneous population of cells (e.g., whole blood or a mixture of cells in a physiological saline solution or physiological medium other than blood). In some aspects, the cell suspension comprises an aqueous solution. In some aspects, the aqueous solution comprises a cell culture medium, PBS, salts, sugars, growth factors, animal derived products, bulking materials, surfactants, lubricants, vitamins, polypeptides, an agent that impacts actin polymerization, or combinations thereof. In some aspects, the cell culture medium comprises DMEM, OptiMEM, EVIDM, RPMI, or combinations thereof. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

When the suspension includes certain types of cells, in some aspects, the cells can be treated with a solution that aids in the delivery of the payload (e.g., reprogramming factor) to the interior of the cell. In some aspects, the solution comprises an agent that impacts actin polymerization. In some aspects, the agent that impacts actin polymerization comprises Latrunculin A, Cytochalasin, Colchicine, or combinations thereof. For example, in some aspects, the cells can be incubated in a depolymerization solution, such as Lantrunculin A, for about 1 hour prior to passing the cells through a constriction to depolymerize the actin cytoskeleton. In some aspects, the cells can be incubated in Colchicine (Sigma) for about 2 hours prior to passing the cells through a constriction to depolymerize the microtubule network.

In some aspects, a characteristic of a cell suspension that can affect the delivery of a payload (e.g., reprogramming factor) into a cell is the viscosity of the cell suspension. As used herein, the term “viscosity” refers to the internal resistance to flow exhibited by a fluid. In some aspects, the viscosity of the cell suspension is between about 8.9×10⁻⁴ Pas to about 4.0×10⁻³ Pa's, between about 8.9×10⁻⁴ Pa's to about 3.0×10⁻³ Pas, between about 8.9×10⁻⁴ Pa's to about 2.0×10⁻³ Pas, or between about 8.9×10⁻⁴ Pas to about 1.0×10⁻³ Pa·s. In some aspects, the viscosity is between about 0.89 cP to about 4.0 cP, between about 0.89 cP to about 3.0 cP, between about 0.89 cP to about 2.0 cP, or between about 0.89 cP to about 1.0 cP. In some aspects, a shear thinning effect is observed, in which the viscosity of the cell suspension decreases under conditions of shear strain. Viscosity can be measured by any suitable method known in the art, including without limitation, viscometers, such as a glass capillary viscometer or rheometers. A viscometer measures viscosity under one flow condition, while a rheometer is used to measure viscosities which vary with flow conditions. In some aspects, the viscosity is measured for a shear thinning solution such as blood. In some aspects, the viscosity is measured between about 0° C. and about 45° C. For example, the viscosity of the cell suspension can be measured at room temperature (e.g., about 20° C.), physiological temperature (e.g., about 37° C.), higher than physiological temperature (e.g., greater than about 37° ° C. to about 45° ° C. or more), reduced temperature (e.g., about 0° C. to about 4° C.), or temperatures between these exemplary temperatures.

### III.B. Payloads

As described herein, in some aspects, a cell suspension additionally comprises one or more payloads (e.g., reprogramming factor). The payloads can be present in the cell suspension prior to, during, and/or after the passing step, in which the cell suspension is passed through the constriction. In some aspects, the cell suspension comprises at least about 2, at least about 3, at least about 4, at least about 5, at least about 6, at least about 7, at least about 8, at least about 9, or at least about 10 or more payloads. As further described elsewhere in the present disclosure, in some aspects, a cell suspension can be passed through multiple constrictions. In such aspects, a payload can be loaded into a cell when the cells pass through one or more of the multiple constrictions. In some aspects, a payload is loaded into a cell each time the cells pass through one or more of the multiple constrictions. When multiple payloads are involved, each of the payloads can be the same. In some aspects, one or more of the payloads are different.

Accordingly, as is apparent from the present disclosure, in some aspects, the squeeze processing methods described herein can be used to deliver multiple payloads to a cell. In some aspects, the multiple payloads can be delivered to a cell using a single squeeze processing (e.g., a cell suspension comprises the multiple payloads, which are delivered to the cell in combination). In some aspects, the multiple payloads can be delivered to a cell repeatedly (e.g., at least two times, at least three times, at least four times, at least five times or more) using the squeeze processing methods described herein. As further described elsewhere in the present disclosure (see, e.g., section titled “Constrictions”), in some aspects, each of the multiple squeeze processing methods can be the same (e.g., same parameters). In some aspects, one or more of the multiple squeeze processing methods can be different (e.g., one or more delivery parameters described herein are different).

As is apparent from the present disclosure, any suitable payloads known in the art can be delivered to a cell using the methods described herein. Non-limiting examples of suitable payloads include a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, DNA comprises a recombinant DNA, a cDNA, a genomic DNA, or combinations thereof. In some aspects, RNA comprises a siRNA, a mRNA, a miRNA, a lncRNA, a tRNA, a shRNA, a self-amplifying mRNA (saRNA), or combinations thereof. In some aspects, the RNA is mRNA. In some aspects, the RNA is siRNA. In some aspects, the RNA is shRNA. In some aspects, the RNA is miRNA. In some aspects, the RNA is a saRNA. In some aspects, a small molecule comprises an impermeable small molecule. As used herein, an “impermeable small molecule” refers to a small molecule that naturally does not cross the cell membrane of a cell

In some aspects, a payload that can be used with the present disclosure comprises a reprogramming factor. In some aspects, the reprogramming factor comprises a differentiation factor. As used herein, the term “differentiation factor” refers to any agent that is capable of inducing the differentiation of a cell into a different type of cell. Unless indicated otherwise, the terms “reprogramming factor” and “differentiation factor” can be used interchangeably in the present disclosure. Accordingly, as used herein, the term “neuron reprogramming factor” or “neuron differentiation factor” can be used interchangeably and refer to an agent that is capable of reprogramming and/or inducing a cell to differentiate into a neuron. As used herein, a neuron reprogramming factor is not particularly limited, as long as the agent is capable of inducing a cell (e.g., stem cell or PBMCs) to differentiate into a neuron. Such neuron reprogramming factors are known in the art. Non-limiting examples of such factors include: neurogenin-2 (Ngn2; Neurog2), Atonal BHLH Transcription Factor 1 (Atoh1), Achaete-Scute Family BHLH Transcription Factor 1 (Ascl1), Nuclear receptor related 1 protein (Nurr1), LIM Homeobox Transcription Factor 1 Alpha (Lmx1a), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124 (mir124), or combinations thereof. In some aspects, the neuron reprogramming factor is Ngn2. In some aspects, the neuron reprogramming factor is Atoh1. In some aspects, the neuron reprogramming factor is Ascl1. In some aspects, the neuron reprogramming factor is Nurr1. In some aspects, the neuron reprogramming factor is Lmx1a. In some aspects, the neuron reprogramming factor is POUR3F2. In some aspects, the neuron reprogramming factor is Myt1l. In some aspects, the neuron reprogramming factor is Foxa2. In some aspects, the neuron reprogramming factor is Pitx3. In some aspects, the neuron reprogramming factor is SOX2. In some aspects, the neuron reprogramming factor is mir124.

As further described and demonstrated herein, such transcription factors can be delivered to a cell (e.g., stem cells or PBMCs) alone or in combination (e.g., at least two, at least three, at least four, at least five, at least six, at least seven, at least about eight, at least about nine, or all of the listed exemplary transcription factors). In some aspects, where combinations of transcription factors (or any other payloads described herein) are involved, they can be delivered to a cell using a single squeeze processing (e.g., concurrent delivery). In some aspects, the combinations of transcription factors can be delivered to a cell (e.g., stem cells or PBMCs) repeatedly. For instance, in some aspects, a combination of transcription factor is delivered to cells with a first squeeze processing; then, the combination of transcription factor is delivered to the cells again with a second squeeze processing. In some aspects, the first squeeze processing includes a microfluidic device (e.g., chip) with multiple rows of constrictions, such that the squeeze process occurs on a single microfluidic device (e.g., chip). As further described herein, in some aspects, the second squeeze processing can occur immediately after the cells have gone through the first squeeze processing (e.g., immediately after the cells pass through the constriction of the first squeeze processing). In some aspects, the second squeeze processing can occur after some time after the first squeeze processing (e.g., at least about 1 minute, at least about 30 minutes, at least about 1 hour, at least about 6 hours, at least about 12 hours, or at least about 1 day after the cells pass through the constriction of the first squeeze processing).

While the above transcription factors generally activate signaling pathways that induce neuron differentiation, in some aspects, neuron reprogramming factors that are useful for the present disclosure can also inhibit signaling pathways, e.g., those pathways that interfere with neuron differentiation. Accordingly, in some aspects, a neuron reprogramming factor comprises a siRNA, such as that can inhibit p53 signaling. In some aspects, a neuron reprogramming factor comprises a miRNA (e.g., miR-214).

In some aspects, the squeeze processing methods described herein can be used to deliver additional compounds, e.g., in combination with the payloads described above. As described elsewhere herein, the squeeze processing method of the present disclosure differs from the more traditional approaches to delivering payloads into cells to induce their reprogramming/differentiation. With the use of viral vectors (e.g., AAV or lentivirus) or with electroporation/lipofection, there are often cytotoxicity and/or homogeneity issues that make such approaches less desirable. With the present methods, as demonstrated herein, there are no lasting negative effects on the cells (e.g., majority of the squeeze-processed cells remain viable and resemble their non-squeeze-processed counterparts). Also, in some aspects, additional compounds can be delivered to cells using the present methods, wherein the additional compounds help improve one or more properties of a mixture comprising the reprogrammed/differentiated cells. In some aspects, the additional compound can comprise a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

For instance, in some aspects, the additional compound is a nucleic acid encoding an enzyme that confers resistance to an antibiotic. Not to be bound by any one theory, by adding these compounds to the cell suspension comprising the plurality of cells and the reprogramming factor, it is possible to increase the purity of a mixture comprising the modified cells (i.e., reprogrammed/differentiated cells). By adding the particular antibiotic associated with the additional compound, the purity of a mixture comprising the modified cells (e.g., comprising differentiated neurons) can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold. Non-limiting example such an additional compound includes puromycin-N-acetyltransferase and the antibiotic is puromycin.

### III.C. Constrictions

### III.C.1. Microfluidic Channels

As described herein, a constriction is used to cause a physical deformity in the cells, such that perturbations are created within the cell membrane of the cells, allowing for the delivery of a payload (e.g., reprogramming factor) into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, the multiple constrictions can be part of a single microfluidic device (e.g., multi-row constriction chip). In some aspects, one or more of the multiple constrictions can be part of different microfluidic devices. For instance, in some aspects, the cells described herein (e.g., stem cells or PBMCs) undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first reprogramming factor, and a second constriction which is associated with a second reprogramming factor, wherein the cell suspension passes through the first constriction such that the first reprogramming factor is delivered to one or more cells of the plurality of cells, and then the cell suspension passes through the second constriction such that the second reprogramming factor is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction. As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, as demonstrated herein (e.g., Example 11), in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than 1 μm. In some aspects the constriction has a length of about 1 μm to about 100 μm. In some aspects, the constriction has a length of less than 1 μm, about 1 μm, about 5 μm, about 10 μm, about 20 μm, about 30 μm, about 40 μm, about 50 μm, about 60 μm, about 70 μm, about 80 μm, about 90 μm, or about 100 μm. In some aspects, the constriction has a length of about 10 μm. In some aspects, the constriction has a length of about 70 μm. In some aspects, the constriction has a depth of about 5 μm to about 90 μm. In some aspects, the constriction has a depth greater than or equal to about 5 μm, about 10 μm, about 20 μm, about 30 μm, about 40 μm, about 50 μm, about 60 μm, about 70 μm, about 80 μm, about 90 μm, about 100 μm, about 110 μm, or about 120 μm. In some aspects, the constriction has a depth of about 10 μm. In some aspects, the constriction has a width of about 3 μm to about 10 μm. In some aspects, the constriction has a width of about 3 μm, about 4 μm, about 4.5 μm, about 5 μm, about 6 μm, about 7 μm, about 8 μm, about 9 μm, or about 10 μm. In some aspects, the constriction has a width of about 6 μm. In some aspects, the constriction has a width of about 6 μm. In some aspects, the constriction has a length of 10 μm, width of 6 μm, and a depth of 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### III.C.2. Surfaces Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### III.D. Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the payloads (e.g., reprogramming factor) described herein to be delivered into the cell. Cells have self-repair mechanisms that allow the cells to repair any disruption in their cell membrane. See Blazek et al., Physiology (Bethesda) 30(6): 438-48 (Nov. 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10-6 second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 us or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻⁸ second. In some aspects, the cell is deformed or perturbed for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 μs, at least about 10 μs, at least about 50 μs, at least about 100 μs, at least about 500 μs, or at least about 750 μs.

In some aspects, the delivery of a payload (e.g., reprogramming factor) into the cell occurs simultaneously with the cell passing through the constriction. In some aspects, delivery of the payload into the cell can occur after the cell passes through the constriction (i.e., when perturbation of the cell membrane is still present and prior to cell membrane of the cells being restored). In some aspects, delivery of the payload into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### III.E. Delivery Parameters

As is apparent from the present disclosure, a number of parameters can influence the delivery efficiency of a payload (e.g., reprogramming factor) into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload (e.g., reprogramming factor) into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described elsewhere in the present disclosure, the payload can be in contact with the population of cells before, during, or after the squeezing step.

In some aspects, by modulating one or more of the delivery parameters, the delivery of the payload (e.g., reprogramming factor) into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload agent into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi.

In some aspects, the particular type of device (e.g., microfluidic chip) can also have an effect on the delivery efficiency of a payload described herein (e.g., reprogramming factor). In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure. In some aspects, the length of the constriction is up to 100 μm. For instance, in some aspects, the length is about 1 μm, about 5 μm, 10 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 3 μm to about 10 μm. In some aspects, the width is about 3 μm, about 4 μm, about 5 μm, about 6 μm, about 7 μm, about 8 μm, about 9 μm, or about 10 μm. In some aspects, the width of the constriction is about 6 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length is about 10 μm, the width is about 6 μm, and depth is about 70 μm.

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload (e.g., reprogramming factor) can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37° ° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### III. F. Therapeutic Uses

In some aspects, the present disclosure relates to the use of the cells produced using the squeeze processing methods described herein to treat various diseases or disorders. As is apparent from the present disclosure, the methods and compositions provided herein can be useful for diseases and disorders where cell replacement therapies can be used as a treatment. By replacing cells that are damaged with the cells produced using the methods provided herein, in some aspects, one or more functions associated with the damaged cells can be restored, and thereby, treat the disease or disorder. For instance, in some aspects, neurons that are produced using the squeeze processing methods provided herein could be administered to a subject suffering from a neurological disorder. The administration of such neurons could be useful in improving one or more symptoms associated with the neurological disorder.

As used herein, the terms “neurological disorder” and “neuroimmunological disorder” can be used interchangeably and refer to diseases and disorders of either the central or peripheral nervous system. Unless specified otherwise, the term “neurological disorders” and “neuroimmunological disorders” comprises all diseases or disorders of the nervous system, including autoimmune disorders. Non-limiting examples of neurological disorders that can be treated with the present disclosure include a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), or combinations thereof. In some aspects, the neurological disorder is Parkinson's disease.

### IV. Compositions of the Disclosures

In some aspects, the disclosure provides a system for delivery of a payload (e.g., reprogramming factor) into a cell, the system comprising a microfluidic channel described herein, a cell suspension comprising a plurality of the cells and the payload; wherein the constriction is configured such that the plurality of cells can pass through the microfluidic channel, wherein the passing of the plurality of cells causes a deformity and disruption of the cell membrane of the cell, allowing the payload to enter the cell.

In some aspects, the disclosure provides a system for delivering a payload, the system comprising a surface with pores, a cell suspension comprising a plurality of the cells and the payload; wherein the surface with pores is configured such that the plurality of cells can pass through the pores, wherein the passing of the plurality of cells causes a deformity and disruption of the cell membrane of the cell, allowing the payload to enter the cell. In some aspects, the surface is a filter or a membrane. In some aspects of the above aspects, the system further comprises at least one electrode to generate an electric field. In some aspects, the system is used to deliver a payload into a cell by any of the methods described herein. The system can include any aspect described for the methods disclosed above, including microfluidic channels or a surface having pores to provide cell-deforming constrictions, cell suspensions, cell perturbations, delivery parameters. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for delivery of a payload (e.g., reprogramming factor) into the cell.

In some aspects, the disclosure provides a cell produced using any of the methods provided herein (e.g., neuron). In some aspects, provided herein is a cell comprising a perturbation in the cell membrane, wherein the perturbation is due to one or more parameters which deform the cell (e.g., delivery parameters described herein), thereby creating the perturbation in the cell membrane of the cell such that a payload (e.g., reprogramming factor) can enter the cell. In some aspects, provided herein is a cell comprising a payload (e.g., reprogramming factor), wherein the payload entered the cell through a perturbation in the cell membrane, which was due to one or more parameters which deform the cell (e.g., delivery parameters described herein) and thereby creating the perturbation in the cell membrane of the cell such that the payload entered the cell. In some aspects, such cells can comprise any of the cells described herein (e.g., stem cells or PBMCs).

In some aspects, the present disclosure provides a composition comprising a plurality of cells, wherein the plurality of cells were produced by any of the methods provided herein. Also provided herein is a composition comprising a population of cells and a payload (e.g., reprogramming factor) under one or more parameters, which result in deformation of one or more cells of the population of cells and thereby creating perturbations in the cell membrane of the one or more cells, and wherein the perturbations in the cell membrane allows the payload to enter the one or more cells.

Also provided are kits or articles of manufacture for use in delivering into a cell a payload (e.g., reprogramming factor) as described herein. In some aspects, the kits comprise the compositions described herein (e.g. a microfluidic channel or surface containing pores, cell suspensions, and/or payload) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The present disclosure also provides kits comprising components of the methods described herein and can further comprise instruction(s) for performing said methods to deliver a payload (e.g., reprogramming factor) into a cell. The kits described herein can further include other materials, including other buffers, diluents, filters, needles, syringes, and package inserts with instructions for performing any methods described herein; e.g., instructions for delivering a payload into a cell.

The following examples are offered by way of illustration and not by way of limitation.

### EXAMPLES

### Example 1: Demonstration of the Delivery of Payload into a Cell

To demonstrate that the methods described herein can be used to deliver a payload into a cell, induced pluripotent stem cells (iPSCs) were treated with Accutase and dissociated into single cells. iPSCs were prepared at a density of 2×10⁷ cells/mL, and dextran (i.e., payload) was added to the cell suspension. Two different sizes of dextran (3 kDa or 70 kDa) were used to assess the relationship between the pressure and the payload size. The cell suspension was added to a constriction channel with the following dimensions at room temperature in either Opti-MEM medium or StemFlex basal medium: length of 10 μm, width of 6 μm, and depth of 70 μm. Cells squeeze processed without any cargo and unprocessed cells were used as controls. The following pressures were tested: 30, 45, 60, 75, or 90 psi. Table 1 (below) provides a summary of the different groups.

TABLE 1

| | | | Delivery | Pres- |
|---|---|---|---|---|
| | sample | Cargo | Buffer | sure |
| 1 | No contact (NC) | None | Opti-MEM | None |
| 2 | Endocytosis | 3 kDa and 70 kDa dextran | Opti-MEM | None |
| | (Endo) | | | |
| 3 | 30 psi | 3 kDa and 70 kDa dextran | Opti-MEM | 30 |
| 4 | 45 psi | 3 kDa and 70 kDa dextran | Opti-MEM | 45 |
| 5 | 60 psi | 3 kDa and 70 kDa dextran | Opti-MEM | 60 |
| 6 | 75 psi | 3 kDa and 70 kDa dextran | Opti-MEM | 75 |
| 7 | 90 psi | 3 kDa and 70 kDa dextran | Opti-MEM | 90 |
| 8 | No contact (NC) | 3 kDa and 70 kDa dextran | StemFlex | None |
| 9 | Endocytosis | 3 kDa and 70 kDa dextran | StemFlex | None |
| | (Endo) | | | |
| 10 | 30 psi | 3 kDa and 70 kDa dextran | StemFlex | 30 |
| 11 | 45 psi | 3 kDa and 70 kDa dextran | StemFlex | 45 |
| 12 | 60 psi | 3 kDa and 70 kDa dextran | StemFlex | 60 |
| 13 | 75 psi | 3 kDa and 70 kDa dextran | StemFlex | 75 |
| 14 | 90 psi | 3 kDa and 70 kDa dextran | StemFlex | 90 |

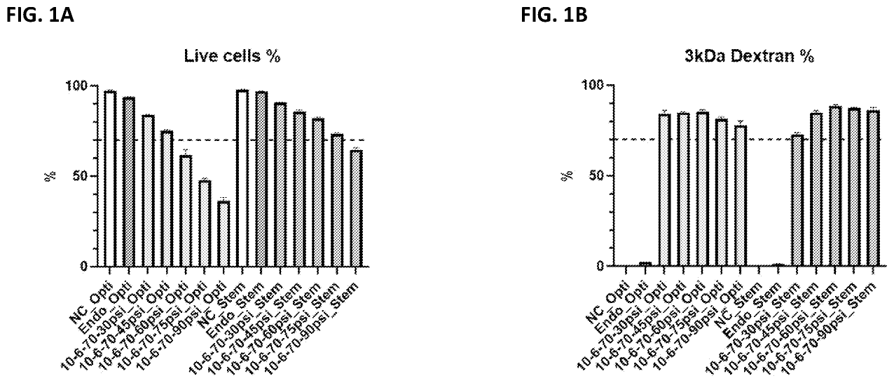

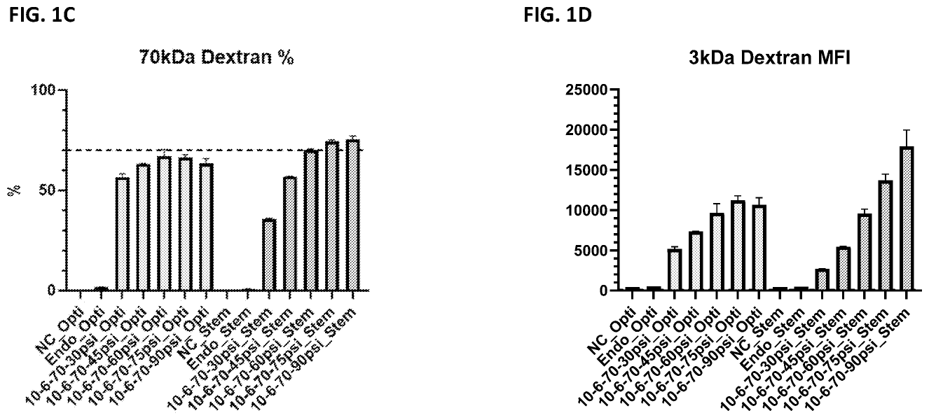

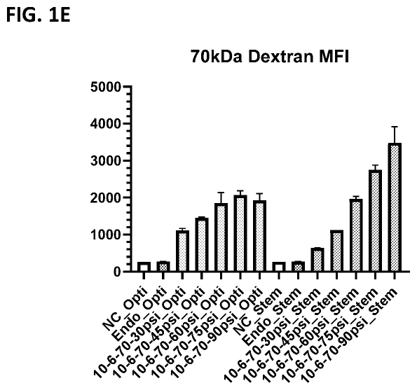

As shown in FIG. 1A, significant percentage of iPSCs remained viable after the squeeze processing under each of the conditions tested. Increased viability of iPSCs were observed with delivery buffer StemFlex basal medium compared to Opti-MEM medium especially at high pressures (e.g., 75 psi and 90 psi). The higher viability corresponded with higher recovery with the StemFlex delivery buffer basal medium using the 70 kDa dextran (FIG. 1C). The 3 kDa dextran delivery rate was similar with both buffers (FIG. 1B). However, the 3 kDa dextran delivered MFI was better using StemFlex basal medium at high pressure conditions (75 and 90 psi) (FIG. 1D).

Overall, these results demonstrate the ability of the squeeze processing methods provided herein to efficiently deliver a payload into a cell.

### Example 2: Analysis of the Effect of Cell Concentration on Payload Delivery Efficiency

To understand the effect that cell concentration has on payload delivery efficiency, iPSCs were treated with Accutase and dissociated into single cells. iPSCs were prepared at the following concentrations: 6×10⁷ cells/mL, 8×10⁷ cells/mL, 1×10⁸ cells/mL, or 1.2×10⁸ cells/mL. The prepared iPSCs were then combined with EGFP mRNA (i.e., payload). The cell suspension was added to the same constriction channel described in Example 1 at room temperature with a pressure of 90 psi. Unprocessed cells were used as controls. Following squeeze-processing, the squeeze-loaded iPSCs were transferred to StemFlex basal medium with supplement and incubated for 24 hours at 37° C. At 24 hours, the cells were collected for analysis of green fluorescent signal by flow cytometry. Table 2 (below) provides a summary of the different groups.

TABLE 2

| | Sample | Cargo | Pressure |
|---|---|---|---|
| 1 | No squeeze | None | None |
| 2 | 60 M/ml | EGFP mRNA | 90 |
| 3 | 80 M/ml | EGFP mRNA | 90 |
| 4 | 100 M/ml | EGFP mRNA | 90 |
| 5 | 120 M/ml | EGFP mRNA | 90 |

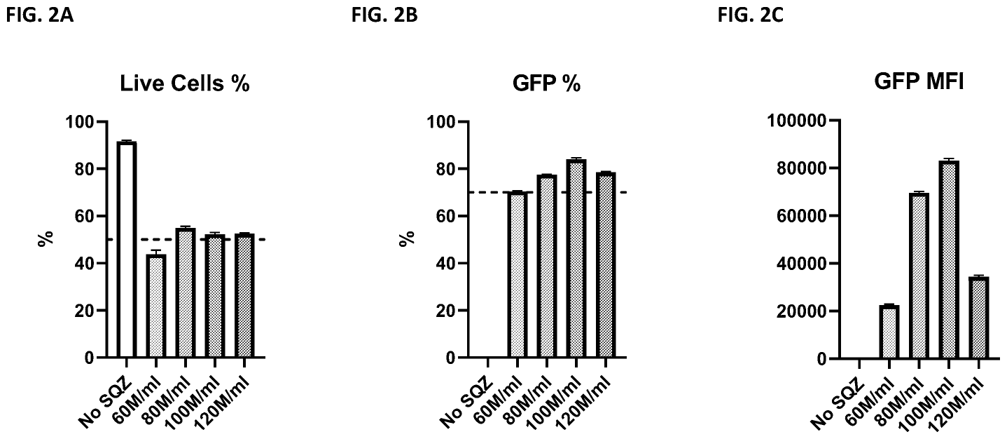

As shown in FIG. 2A, at most of the cell concentrations tested, at least about 50% of the squeeze-loaded iPSCs remained viable. With all the cell concentration tested, at least about 70% of the squeeze-loaded iPSCs expressed GFP fluorescent protein (FIG. 2B). However, as shown in FIG. 2C, at the highest cell density tested (120M/ml), there was significantly less EGFP mRNA loaded on a cell per basis, suggesting a relationship between cell density and the amount of payload that is loaded into the cells. As to cell viability and the percentage of cells that are loaded with the payload, cell density had minimal effect.

### Example 3: Analysis of the Effect of Pressure on Payload Delivery Efficiency

To understand the effect that pressure (i.e., the pressure at which the cell suspension is pushed through the constriction) has on payload delivery efficiency, iPSCs were treated with Accutase and dissociated into single cells. iPSCs were prepared at a density of 2×10⁷ cells/mL and combined with mRNA encoding Cy5 EGFP. The cell suspension was added to the same constriction channel described in Example 1 at room temperature using one of the following pressure: 45 psi, 60 psi, or 75 psi. No squeeze processed cells incubated with Cyanine 5 EGFP mRNA was used as a control. Following squeeze-processing, the squeeze-loaded iPSCs were collected for analysis Cyanine 5 fluorescent signal by flow cytometry. Table 3 (below) provides a summary of the different groups.

TABLE 3

| | sample | Cargo | Pressure |
|---|---|---|---|
| 1 | No contact (NC) | None | None |
| 2 | Endocytosis (Endo) | Cyanine 5 EGFP mRNA | None |
| 3 | 45 psi | Cyanine 5 EGFP mRNA | 45 |
| 4 | 60 psi | Cyanine 5 EGFP mRNA | 60 |
| 5 | 75 psi | Cyanine 5 EGFP mRNA | 75 |

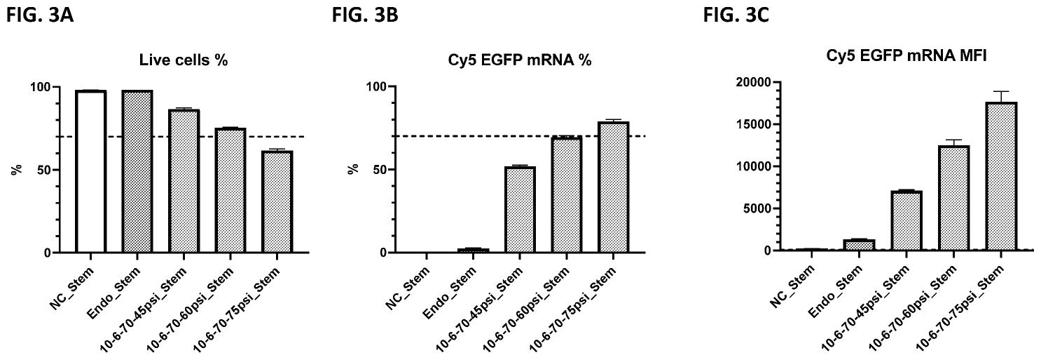

As shown in FIG. 3A, there was a modest decrease in viability at the higher pressure. However, the delivery of the EGFP mRNA was greatly improved with higher pressure. As shown in FIGS. 3B and 3C, the greatest Cyanine 5 fluorescent signaling and the highest Cyanine 5 MFI was detected at 75 psi. These results demonstrate that the delivery efficiency of a payload can be regulated with modulation of the pressure.

### Example 4: Analysis of iPSC Phenotype after Squeeze Processing

As described herein, as cells pass through a constriction, they are physically deformed, which results in the perturbation of the cell membrane. While the perturbation is temporary, additional analysis was conducted whether there were any alterations as to the phenotype of the cell. Briefly, iPSCs were again treated with Accutase and dissociated into single cells. Then, the iPSCs were prepared at a density of 1×10⁸ cells/mL, and passed through the same constriction channel as that described in Example 1 at room temperature with a pressure of 75 psi. No payloads were involved. Also, no squeeze processed cells were used as control. The iPSCs that passed through the constriction were transferred to StemFlex basal medium with supplement and incubated for 24 hours at 37° C. After the incubation, the expression of several iPSC pluripotent markers (e.g., Oct4, Nanog, Sox2, MYC, TERT, and/or SSEA4) was assessed by RT-qPCR and flow cytometry. The expression of 32 different housekeeping genes was also assessed using RT-qPCR.

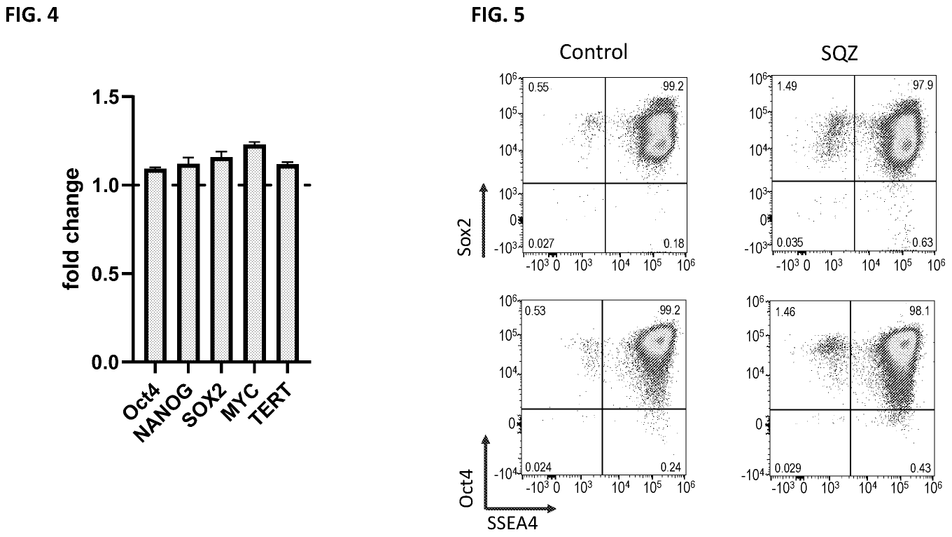

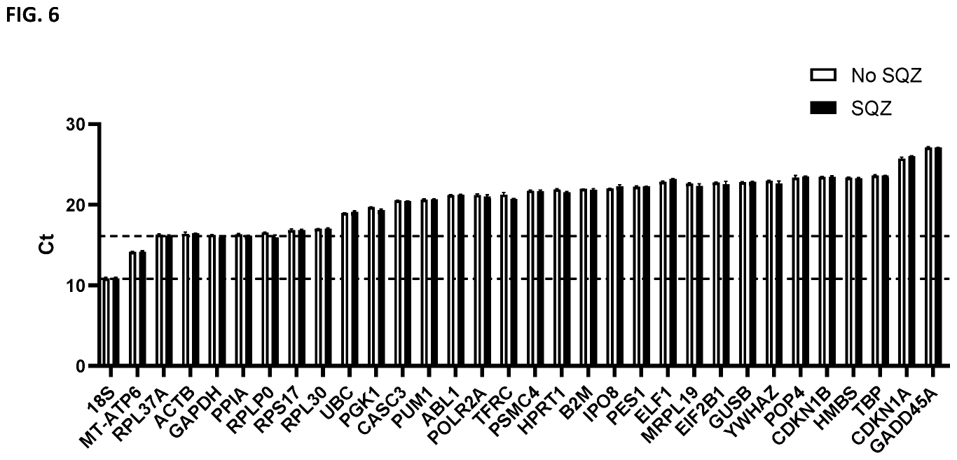

As shown in FIGS. 4-6, there was minimum fold change in the expression of various genes assessed between no contact and squeeze processed iPSCs after 24 hours. Such data indicates that the squeeze process described herein does not alter the phenotype of the cells.

Collectively, the above results demonstrate that the delivery methods described herein (i.e., squeeze process) is capable of delivering a payload into cells without any negative effects to the cells.

### Example 5: Use of Ngn2 to Induce Neuron Differentiation

As described herein, various cells are capable of being reprogrammed to different types of cells, e.g., through the delivery of one or more reprogramming agents into the cells. Such ability to reprogram cells to a particular cell type of interest could be useful in the treatment of various diseases and disorders, particularly in the context of regenerative medicine and adoptive cellular therapy. Accordingly, to assess the efficiency of the methods described herein in inducing the differentiation of cells into neurons, five different mRNAs encoding the neurogenin-2 protein were constructed: (1) SA-Ngn2 mRNA (comprises a serine to alanine mutation); (2) SA-co-Ngn2 mRNA (codon optimized); (3) Sa-co-m6AG-Ngn2 mRNA (lower decapping rate); (4) SA-co-KOCO-Ngn2 mRNA (comprises 10 lysine to arginine mutations and 6 cysteine to alanine mutations to block ubiquitination); and (5) SA-co-Kozak-Ngn2 mRNA (second amino acid is mutated to alanine to fit Kozak consensus sequence). The different Ngn2 mRNAs were then added to a cell suspension comprising iPSCs (at a density of 1×10⁸ cells/mL) that were treated with Accutase and dissociated into single cells. The cell suspensions were added to constrictions with the same dimensions as that described in Example 1 at room temperature with a pressure of 90 psi. Squeeze processed cells without cargo were used as control. After the cells had passed through the constriction, the cells were collected and transferred to StemFlex basal medium with supplement and incubated for 8 hours at 37° C. Then, the expression of the Ngn2 protein was assessed in the cells using Western blot analysis.

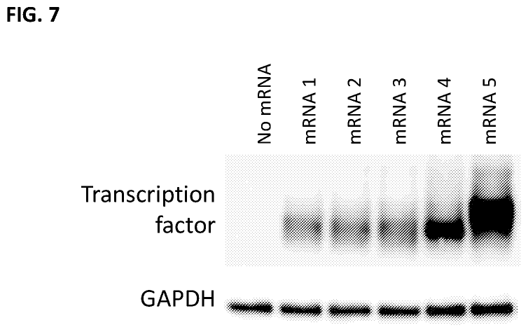

As shown in FIG. 7, varying levels of the Ngn2 protein were observed in the different iPSCs, suggesting that each of the Ngn2 mRNAs were delivered to the iPSCs to some degree. SA-co-Kozak Ngn2 mRNA resulted in the most robust and durable protein expression at 8 hours after squeeze delivery.

### Example 6: Analysis of Ngn2-Derived Neurons after Squeeze Processing

To assess whether the delivery of Ngn2 mRNA to cells using squeeze processing methods described herein can induce neuron differentiation, iPSCs were treated with Accutase and dissociated into single cells. Then, the iPSCs were prepared at a density of 1×10⁸ cells/mL and combined in a cell suspension with one of the following Ngn2 mRNA constructs: (1) SA-co-Ngn2 mRNA (codon optimized); (2) Sa-co-m6AG-Ngn2 mRNA (lower decapping rate); (3) SA-co-m6AG-DBG-Ngn2 mRNA (DBG: double human β-globin 3′UTR); (4) SA-co-Kozak-Ngn2 mRNA second amino acid is mutated to alanine to fit Kozak consensus sequence); and (5) SA-co-Kozak-AES-Ngn2 mRNA (AES: AES-mtRNR1 3′UTR, AES: Amino-terminal enhancer of split, mtRNR1: Mitochondrially encoded 12S rRNA). The cell suspensions were added to constrictions with the same dimensions as that described in Example 1 at room temperature with a pressure of 90 psi. Squeeze processed cells without cargo were used as control. After the cells had passed through the constriction, the cells were collected and transferred to StemFlex basal medium and incubated at 37° C. At 12 and 24 hours post incubation, cells were washed with PBS to remove any debris, and the expression of NeuroD1 and NeuroD4 (downstream target genes of Ngn2) was assessed using RT-qPCR.

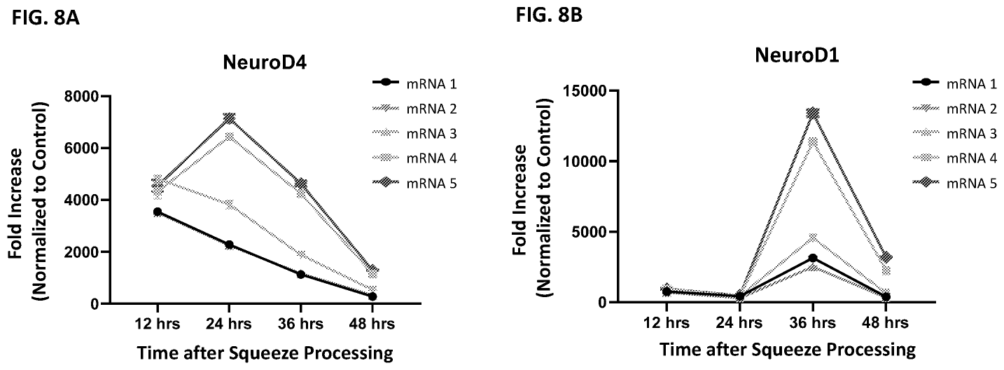

As shown in FIGS. 8A and 8B, Ngn2 downstream target genes NeurD4 and NeuroD1 had different kinetics expression profile. NeuroD4 started to express at 12 hours, reached peak expression at 24 hours, and was back to baseline by 48 hours. NeuroD1 reached peak expression at 36 hours and remained elevated at 48 hours. SA-co-Kozak Ngn2 and SA-co-Kozak-AES Ngn2 mRNA induced the highest Ngn2 downstream targets NeuroD4 and NeuroD1 expression. There was no difference in NeuroD4 and NeruoDI expression between SA-co Ngn2 and SA-co-m6AG Ngn2, suggesting that m6AG 5′ capping is not improving the stability of mRNA.

### Example 7: Analysis of the Use of Puromycin to Increase the Purity of Composition Comprising Ngn2-Derived Neurons

As described herein and demonstrated in the earlier examples, one or more parameters of the squeezing process can be modulated to increase the delivery efficiency of a payload into a cell. To identify additional approaches to improving the purity of the cells collected after the squeeze process, iPSCs were treated with Accutase and dissociated into single cells. iPSCs were prepared at a density of 1×10⁸ cells/mL, and were combined with one of the following payloads: (1) none; (2) SA-co-Kozak-AES-Ngn2 mRNA (AES: AES-mtRNR1 3′UTR, AES: Amino-terminal enhancer of split, mtRNR1: Mitochondrially encoded 12S rRNA); (3) co-PAC-mRNA (codon-optimized mRNA encoding puromycin N-acetyltransferase); or (4) combination of SA-co-Kozak-AES-Ngn2 and co-PAC mRNA (7:3 ratio). The cell suspensions were added to constrictions with the same dimensions as that described in Example 1 at room temperature with a squeeze of 90 psi. Squeeze processed cells without cargo were used as control.

Following squeeze-processing, the squeeze-loaded iPSCs were transferred to StemFlex basal medium with supplement and incubated for 6 hours at 37° C. At 6 hours, wash cells with PBS to get rid of debris and add 1:1 ratio of StemFlex basal medium with supplement/N2B medium+B27 (100×) with and without 5 μg/ml puromycin. Phase contrast images were taken at 20 hours post squeeze delivery.

Additionally, as further characterization, some of the cells that passed through the constriction were fixed and the expression of Nanog (iPSC marker) and Tuj1 (early neuronal marker) were assessed using immunofluorescence microscopy.

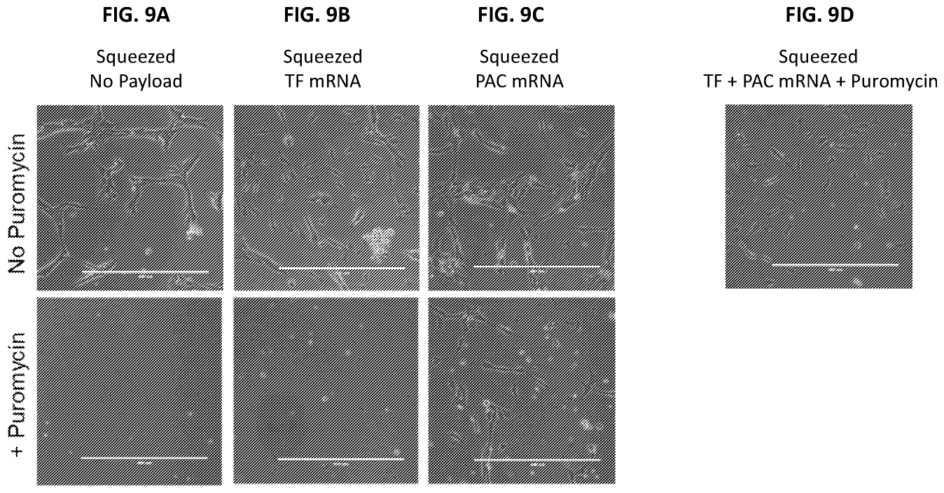

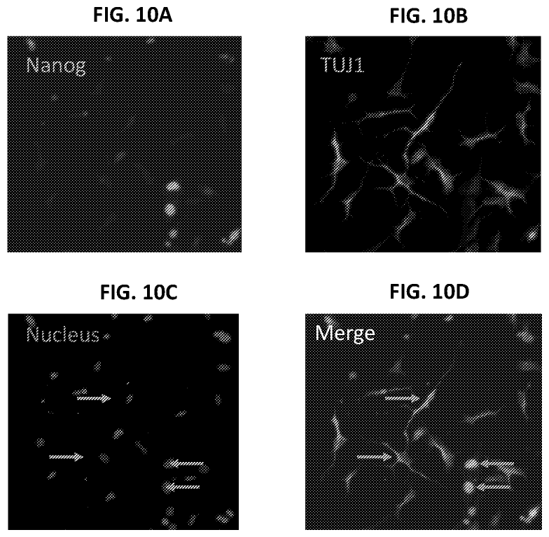

As shown in FIGS. 9A-9D, cells delivered with PAC mRNA were resistant to puromycin treatment. 12 hours of puromycin treatment was sufficient to kill all the cells without PAC mRNA delivery. Codelivery of Ngn2 and PAC mRNA with 12 hours of puromycin treatment got rid of most but not all iPSCs. And, as shown in FIGS. 10A-10D, Nanog and Tuj1 antibodies successfully stained iPSCs and Ngn2-derived neurons respectively. After Ngn2 mRNA delivery, cells were either stained with Nanog or Tuj1, suggesting a rapid cell fate transition from iPSC to neuron stage. Early neuronal marker Tuj1 could be detected as early as day 1 after squeezed delivery. (FIG. 10B).

To confirm the above results, some of the cells that passed through the constriction were treated with puromycin for 12 hours, and then additionally treated with AraC for 4 days to remove non-neuronal cells. Then, at 7 days after the cells had passed through the constriction, the cells were fixed and the expression of both Tuj1 (early neuronal marker) and MAP2 (mature neuronal marker) were assessed.

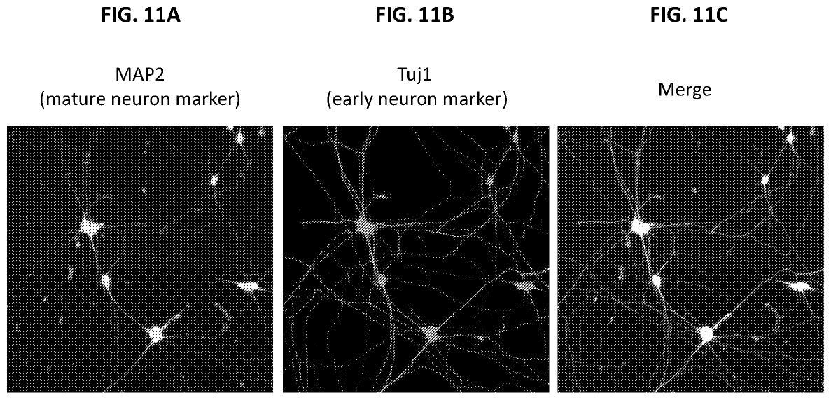

As shown in FIGS. 11A-11E, at day 7, Ngn2-derived neurons expressed high MAP2 and Tuj1 expression, demonstrating the successful differentiation of the iPSCs to neurons using the squeeze processing methods provided herein.

### Example 8: Analysis of Pressure Sweep to Maximize Neuron Differentiation

To help further maximize the production of Ngn2-derived neurons, cell suspensions comprising iPSCs that were treated with Accutase and dissociated into single cells (1×10⁸ cells/mL) and combination of SA-co-Kozak-AES-Ngn2 and co-PAC mRNA (7:3 ratio). The cell suspensions were added to constrictions with the same dimensions as that described in Example 1 at room temperature using one of the following pressures: 30 psi, 45 psi, 60, psi, or 75 psi. Squeeze processed cells without cargo was used as a control. Following squeeze-processing, the squeeze-loaded iPSCs were transferred to StemFlex basal medium with supplement and incubated for 6 hours at 37° C. At 6 hours, wash cells with PBS to get rid of debris and add 1:1 ratio of StemFlex basal medium with supplement/N2B medium+B27 (100×). At 24 hours, cells were collected and stained for iPSC pluripotent marker Nanog and early neuronal marker Tuj1 for flow cytometry analysis. Table 4 (below) provides a summary of the different treatment groups.

TABLE 4

| | sample | Cargo | Pressure |
|---|---|---|---|
| 1 | Squeeze no cargo | None | 30 |
| 2 | Ngn2 + PAC (30 psi) | SA-co-Kozak-AES Ngn2 and | 30 |
| | | co-PAC mRNA (7:3) | |
| 3 | Ngn2 + PAC (45 psi) | SA-co-Kozak-AES Ngn2 and | 45 |
| | | co-PAC mRNA (7:3) | |
| 4 | Ngn2 + PAC (60 psi) | SA-co-Kozak-AES Ngn2 and | 60 |
| | | co-PAC mRNA (7:3) | |
| 5 | Ngn2 + PAC (75 psi) | SA-co-Kozak-AES Ngn2 and | 75 |
| | | co-PAC mRNA (7:3) | |

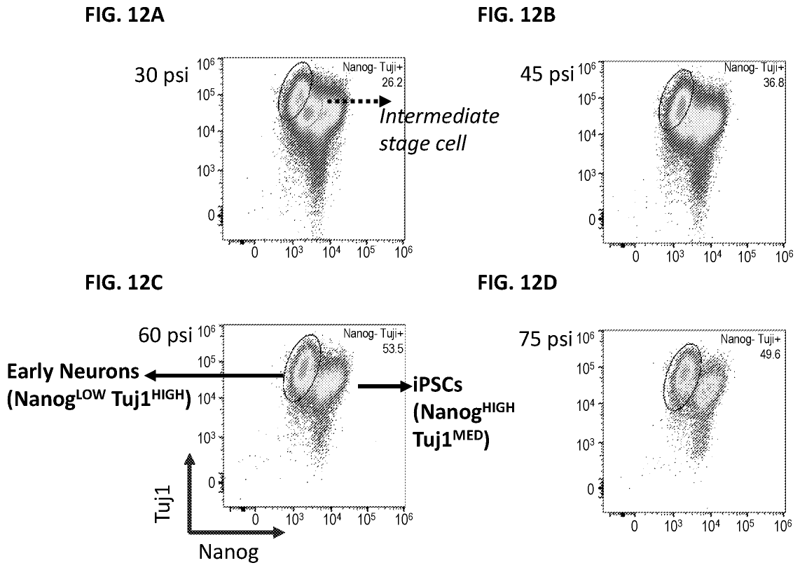

As shown in FIGS. 12A-12D, higher pressure (60 and 75 psi) generated similar percentage of early neurons (Nanog^{LOW} with Tuj1^{HIGH}) while 60 psi have higher cell retention. Lower pressure (30 and 45 psi) created an intermediate stage cell between early neurons (Nanog^{LOW} with Tuj1^{HIGH}) and iPSC (Nanog^{HIGH} with Tuj1^{MED}), suggesting insufficient Ngn2 expression.

### Example 9: Analysis of the Relationship Between Ngn2 Delivery Efficiency and Neuron Generation

To determine if more Ngn2 delivery correlated with earlier neuron generation, Ngn2_T2A_GFP mRNA or Ngn2 with GFP mRNA were delivered into iPSCs using the methods described herein. Ngn2_T2A_GFP is a single mRNA construct which Ngn2 and GFP nucleotide sequence is linked by a T2A self-cleavage sequence. The delivery of Ngn2_T2A_GFP mRNA into iPSCs will allow us to quantify the amount of Ngn2 with the GFP fluorescent protein signaling with flow cytometry. Ngn2 and GFP were also co-delivered as two separate mRNA construct for comparison. Specifically, iPSCs were treated with Accutase and dissociated into single cells. iPSCs were prepared at a density of 1×10⁸ cells/mL and combined with the different payloads. The cell suspensions were added to constrictions with the same dimensions as that described in Example 1 at room temperature using one of the following pressures: 45 psi, 60 psi, or 75 psi. No squeeze processed cells was used as a control. Table 5 (below) provides a summary of the different groups.

Following squeeze-processing, the squeeze-loaded iPSCs were transferred to StemFlex basal medium with supplement and incubated for 6 hours at 37° C. At 6 hours, wash cells with PBS to get rid of debris and add 1:1 ratio of StemFlex basal medium with supplement/N2B medium+B27 (100×). At 24 hours, cells were collected and stained for iPSC pluripotent marker Nanog for flow cytometry analysis.

TABLE 5

| | sample | Cargo | Pressure |
|---|---|---|---|
| 1 | No Squeeze | None | None |
| 2 | Ngn2_T2A_GFP (45 psi) | SA-co-Kozak-Ngn2_T2A_GFP mRNA | 45 |
| 3 | Ngn2_T2A_GFP (60 psi) | SA-co-Kozak-Ngn2_T2A_GFP mRNA | 60 |
| 4 | Ngn2_T2A_GFP (75 psi) | SA-co-Kozak-Ngn2_T2A_GFP mRNA | 75 |
| 5 | Ngn2 + GFP (45 psi) | SA-co-Kozak-Ngn2 + GFP mRNA (7:3) | 45 |
| 6 | Ngn2 + GFP (60 psi) | SA-co-Kozak-Ngn2 + GFP mRNA (7:3) | 60 |
| 7 | Ngn2 + GFP (75 psi) | SA-co-Kozak-Ngn2 + GFP mRNA (7:3) | 75 |

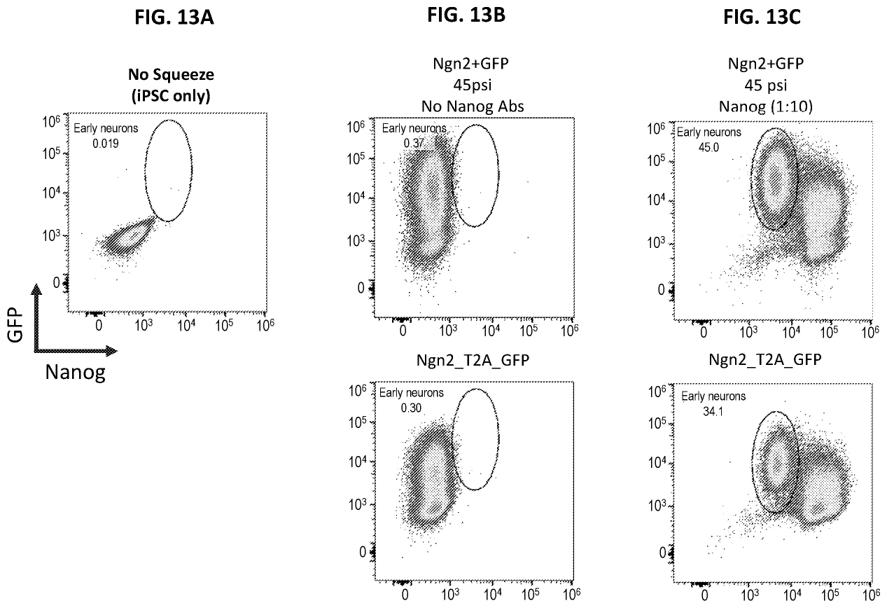

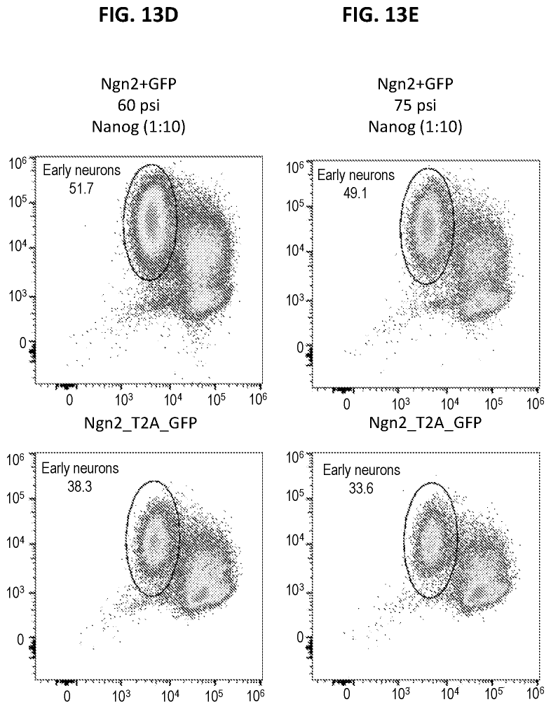

The amount of Ngn2 was quantified with GFP fluorescent protein signaling and used the loss of Nanog which is a pluripotent marker for early neuron production. As shown in FIGS. 13A-13E, there was an inverse correlation between Ngn2 (GFP) and Nanog expression indicating that the higher Ngn2 (GFP) delivered, the earlier the neuron generated (the loss of Nanog expression.) Ngn2 with GFP mRNA generated more early neurons compared to a single Ngn2_T2A_GFP mRNA. The most likely explanation is that because a single Ngn2_T2A_GFP mRNA is larger compared to either Ngn2 or GFP mRNA. Because squeeze delivery is based on diffusion, there were more Ngn2 mRNA codelivered with GFP mRNA compared to a single Ngn2_T2A_GFP mRNA.

### Example 10: Use of Atoh1 to Induce Neuron Differentiation

To assess whether other reprogramming agents can be used to induce neuron differentiation, the following mRNA constructs were used as payloads: (1) WT-Atoh1 mRNA (encoding the wild-type Atoh1); (2) SA-Atoh1 mRNA (encoding Atoh1 with serine to alanine mutation: S331A and S342A); (3) SA-co-Atoh1 mRNA (codon optimized); or (4) SA-co-Kozak-Atoh1 mRNA (second amino acid mutated to Alanine to fit the consensus Kozak sequence). The different Atoh1 mRNA constructs were then added to a cell suspension comprising iPSCs (at a density of 1×10⁸ cells/mL) that were treated with Accutase and dissociated into single cells. The cell suspensions were added to constrictions with the same dimensions as that described in Example 1 at room temperature with a pressure of 60 psi. Squeeze processed cells without cargo were used as control.

Following squeeze-processing, the squeeze-loaded iPSCs were transferred to StemFlex basal medium with supplement and incubated for 6 hours at 37° ° C. At 6 hours, wash cells with PBS to get rid of debris and add 1:1 ratio of StemFlex basal medium with supplement/N2B medium+B27 (100×). Cell lysates were collected at 6 and 20 hours after SQZ delivery for Western blot. Total RNAs were collected at 12 and 24 hours after SQZ delivery for RT-qPCR.

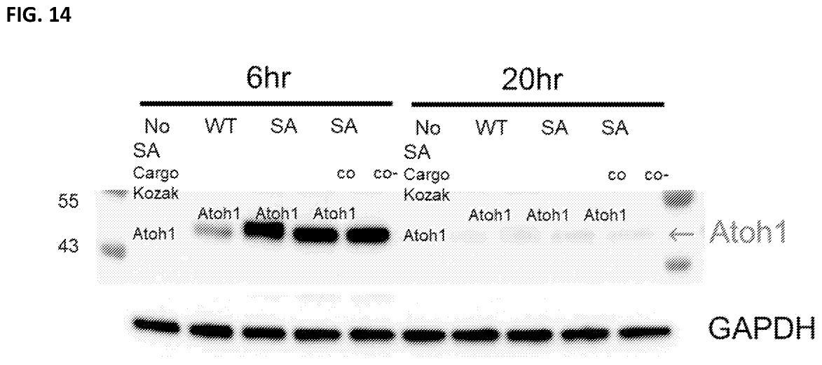

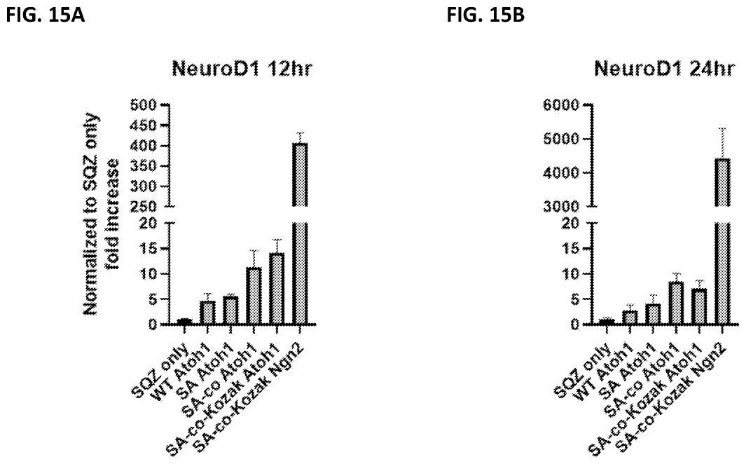

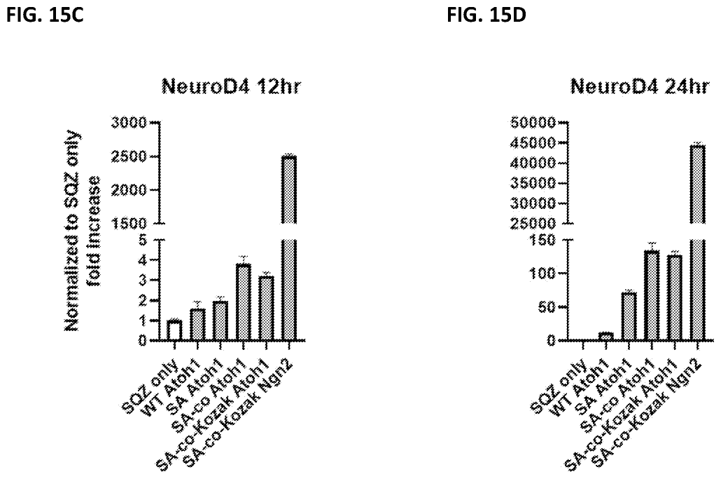

As shown in FIG. 14, SA Atoh1, SA-co Atoh1, and SA-co-Kozak Atoh1 mRNAs expressed robust Atoh1 proteins at 6 hr and diminished at 20 hr after squeezed delivery. SA-co and SA-co-Kozak Atoh1 induced Atoh1 downstream genes NeuroD1 and NeuroD4 the most, indicating that fitting Kozak sequence did not further increase the Atoh1 expression (FIGS. 15A-15D). The level of downstream gene expressions induced by Atoh1 were low compared to the induction by SA-co-Kozak Ngn2.

### Example 11: Analysis of the Effect of Repeated Squeeze-Delivery of Multiple Payloads

This study evaluated the cell viability and recovery, as well as the activation of endogenous dopaminergic neuronal gene expression in freshly isolated human bulk PBMCs upon repeated multiplex squeeze-delivery (i.e., delivery of multiple payloads). Specifically, synthetic mRNAs encoding for reprogramming factors, NURR1, FOXA2, LMX1A, BRN2, ASCL1, MYT1L, NGN2, and SOX2 (NFL-BAMN-S) were used as payloads. PBMCs were repeatedly delivered (i.e., at t=0 and t=24 hours) either with four neurogenic factors, BRN2, ASCL1, MYT1L, NGN2 (BAMN) or the full set of eight factors (NFL-BAMN-S). As a control, PBMCs with no squeeze (no contact) or squeeze processed with no cargo (empty squeeze) were used.

### Methods

Day 0 (first squeeze; i.e., t=0): Human PBMCs were isolated from a leukopak. Cells were prepared at a cell concentration of 2×10⁸ cells/mL in RPMI (2×). A medium of R10 (RPMI 1640+10% fetal bovine serum+100 U/mL penicillin+100 μg/mL streptomycin) was prepared.

A cocktail of four mRNAs (BAMN) or eight mRNAs (NFL-BAMN-S) was prepared in RPMI media at a concentration of 500 μg/mL for each mRNA (2×). Final volume of 300 μl was prepared for each mRNA mix. NaCl (0.154M) was added to the mRNA mix to adjust the tonicity. The mRNA mix was left undisturbed for 30 min at room temperature prior to squeezing. The following groups were used in the study. Table 6 (below) describes the different groups:

TABLE 6

| | | | Number of |
|---|---|---|---|
| | Squeeze | | Squeeze |
| Group | Condition | Squeeze Volume | per Chip |
| A | No Contact | 200 μl (100 ul cells + 100 ul media) | 3x |
| B | Empty | 200 μl (100 ul cells + 100 ul media) | 3x |
| | Squeeze | | |
| C | BAMN | 200 μl (100 ul cells + 100 ul mRNA) | 3x |
| D | NFL- | 200 μl (100 ul cells + 100 ul mRNA) | 3x |
| | BAMN-S | | |

100 μl cells (20 million) and 100 μl mRNA solution were mixed at 1:1 ratio immediately prior to squeezing. The cells were first squeeze processed (i.e., t=0) (over three rounds: 200 μl/round for a total of 600 μl of cell-mRNA mix) at room temperature using a microfluidic constriction (10 μm depth, 3.5 μm width, and 70 μm length) at 60 psi in RPMI medium. Cell viability and recovery analysis were done before and after the squeeze using Nucleocounter NC-200 and Orflo Moxi Go II Cell Counter. Squeezed cells were pooled together, spun at 500 g for 5 min at room temperature, and cultured at a concentration of 2 million cells/mL, and cultured in R10 media overnight at 37° ° C., 5% CO2 in 75 cm2 flasks. Subset of cells were cultured in 96-well round bottom plates (2 million/mL; 400×10⁵ cells/200 μl/well) for qPCR analysis at 3 hours- and 24-hours-post-delivery.

Day 1 (second squeeze; i.e., t=24): After 24 hours of incubation, cells (after the first squeeze) were harvested into a 50 ml tube using a 10 mL serological pipette by pipetting up and down two times gently and spun at 400 g for 4 min at room temperature. In parallel, to dislodge the remaining attached cells, flasks were first washed with sterile PBS, then treated with the Accutase solution for 3 min. The dislodged cells were transferred to a 15 mL tube and spun for 4 min at 400 g at room temperature. Cells from the two spins were pooled together in a new 15 mL tube, spun again for 4 min at 400 g at room temperature. Final cell concentration was adjusted to 1×10⁸ cells/mL in RPMI (half the concentration of the first squeeze). mRNA solutions were prepared at the same concentration and volume as the first squeeze (500 μg/mL from each mRNA for 2× mix).

50 mL neuronal differentiation medium was prepared comprising DMEM/F12 (48 mL)+B27-plus (1×)+N2 (1×)+100 U/mL penicillin (0.5 mL), supplemented with small molecules forskolin (cAMP activator) (3 μM), dorsomorphin (BMP blocker) (2 μM), and SB431542 (TGF-B blocker) (10 μM).

The cells were squeeze processed for the second time (over two rounds: 200 μl/round) using the same parameters with the first squeeze (using a microfluidic constriction (10 μm depth, 3.5 μm width, and 70 μm length) at 60 psi in RPMI medium at room temperature). Cell viability and recovery analysis were again done before and after the squeeze using Nucleocounter NC-200 and Orflo Moxi Go II Cell Counter. The squeezed cells were incubated in 96-well plates in neuronal differentiation media with small molecules in 96-well round-bottom plates (2 million/mL; 400 K cells/200 μl/well) at 37° C., 5% CO2 for 48 hours. Cell viability was measured at 48 hours-post-second-squeeze using Nucleocounter NC-200 and Orflo Moxi Go II Cell Counter.

Cells were harvested for RNA extraction and qPCR analysis at 3 hours first and second squeeze. RNA isolation and cDNA synthesis were done using Qiagen RNeasy Mini Plus kit and Qiagen QuantiTect reverse transcription kits, respectively. qPCR analyses of Tubb3 (Tuj1) and Nr4a2 (Nurr1) were done using ThermoFisher TaqMan gene expression system (Tuj1, Hs00964962_g1; Nurr1, Hs00428691_ml).

### Results

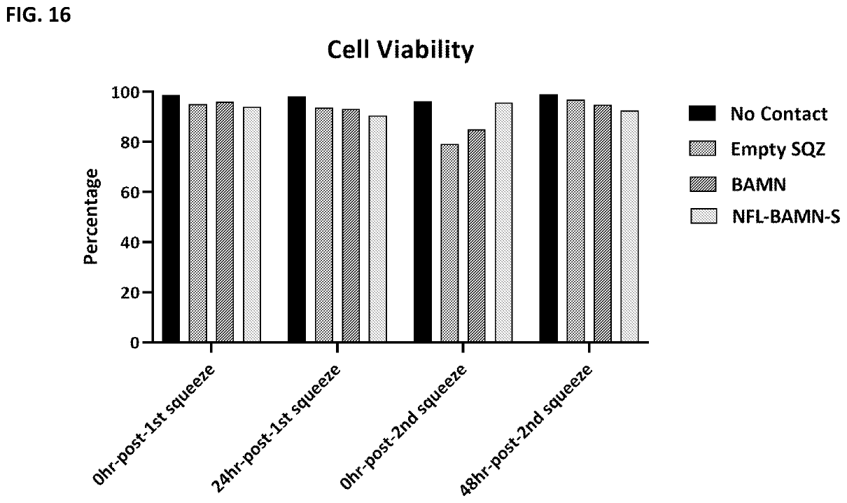

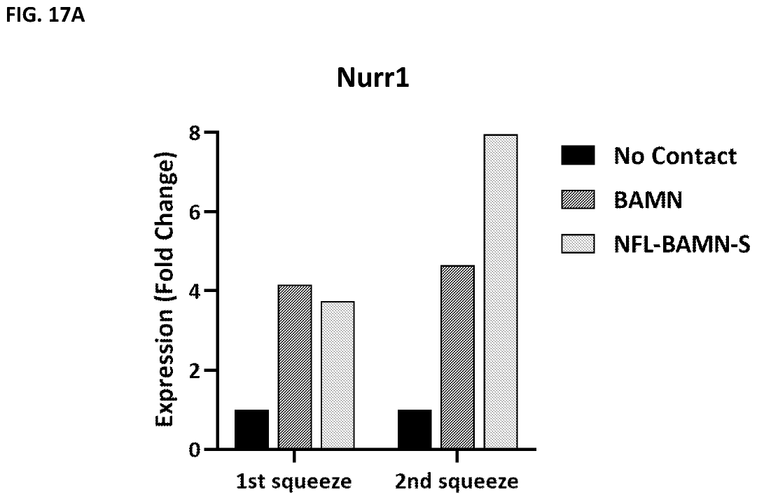

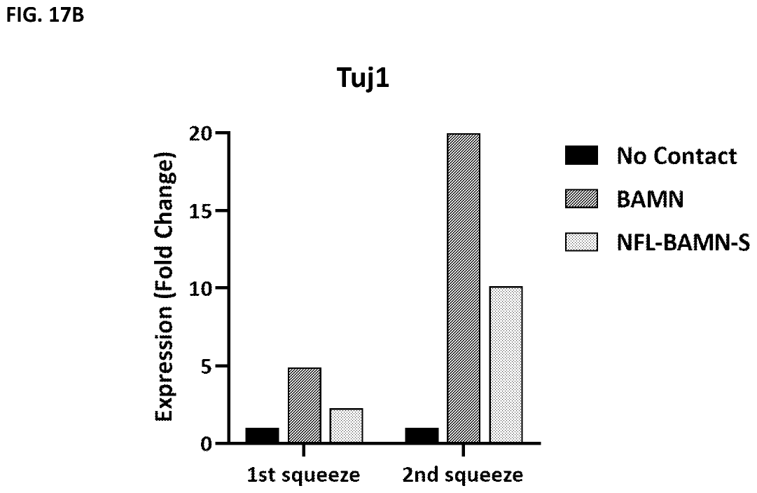

As shown in FIG. 16, in all groups tested, cell viability was comparable after the first and second squeeze processing. For instance, approximately >90% of the cells from all the groups remained viable after the second squeeze processing. Additionally, the data also showed that PBMCs can be cultured in neuronal media over 2-3 days without a noticeable impact on cell survival. And, as shown in FIGS. 17A and 17B, endogenous expression of dopaminergic neuronal molecules Tuj1 and Nurr1 were activated in the PBMCs upon-repeated delivery of the eight reprogramming factors (see NFL-BAMN-S group). Collectively, the above results further demonstrate that the squeeze processing methods described herein can be effectively used to repeatedly deliver multiple payloads to PBMCs, and thereby, inducing the differentiation of the PBMCs to neurons.

### Example 12: Analysis of Neuronal and Dopamine Lineage Markers after Ascl1, Lmx1a, Nurr1, and FoxA2 mRNA Delivery Using Squeeze Processing

To assess whether the delivery of mRNAs encoding certain reprogramming factors to cells using squeeze processing methods described herein can induce dopaminergic neuron differentiation, iPSCs were treated with Accutase and dissociated into single cells. The iPSCs were prepared at a density of 1×10⁸ cells/mL, and a cocktail of codon-optimized mRNAs encoding Ascl1, Lmx1a, Nurr1, FoxA2, and PAC was added to the cell suspension containing the prepared iPSCs. The cells (i.e., iPSCs) were squeeze processed at room temperature using a microfluidic constriction (length of 10 μm, width of 6 μm, and depth of 70 μm) at 60 psi. Cells squeeze processed without any cargo were used as controls.

Following squeeze processing, the cells that passed through the constriction were collected and transferred to StemFlex basal medium with supplement and incubated for 6 hours at 37° C. At 6 hours, the cells were washed with PBS to remove unwanted debris. A mixture of 1:1 ratio of StemFlex basal medium with N2B medium+B27 (100×) with 5 μg/ml puromycin was added. The mixture was incubated for 18 hours. The medium was then removed, and a mixture of NBM medium+B27 (50×)+BDNF+GDNF+CNTF was added at a concentration of 1:1000. Total RNA was collected from the cells at 8, 24, and 48 hours, and RT-qPCR was used to analyze the expression of general neuronal markers (NeuroD1, and Neurog2) and dopamine lineage markers (FoxA2, Pitx3, Lmx1a, and TH).

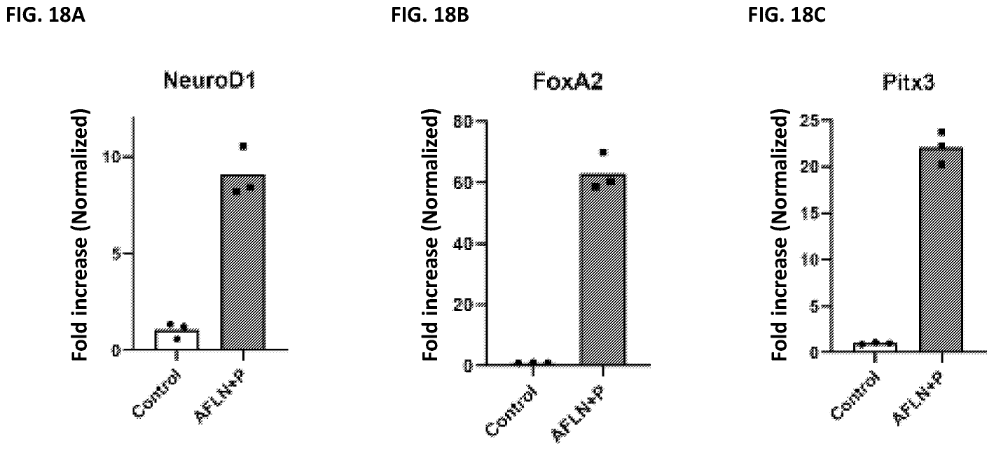

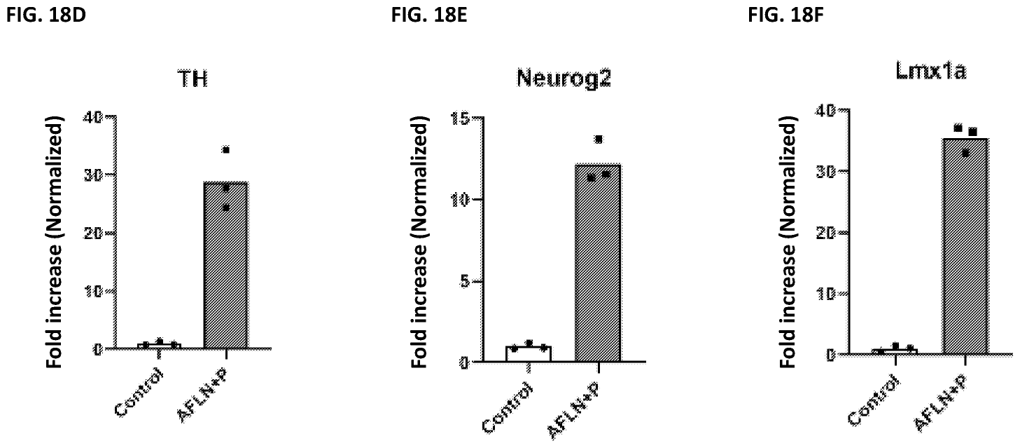

As show in FIGS. 18A-18F, the combined delivery of Ascl1, Nurr1, Lmx1a, and FoxA2 mRNAs using squeeze processing induced both neuronal and dopaminergic neuron lineage marker expression in the iPSCs. For instance, at 8 hours post squeeze processing, there was significantly higher expression of neuroD1, Foxa2, and Pitx3 genes in the squeeze processed iPSCs compared to the control cells (FIGS. 18A, 18B, 18C, respectively). Similarly, at 24 hours, the squeeze processed iPSCs expressed significantly higher level of the TH gene (FIG. 18D). And, at 48 hours post squeeze processing, the squeeze processed iPSCs expressed much higher levels of both Neurog2 and Lmx1a genes compared to the control cells (FIGS. 18E and 18F, respectively).

### Example 13: Analysis of Ascl1, Lmx1a, Nurr1, and FoxA2 Protein Expression after Squeeze Processing Delivery of mRNAs

To assess whether there was also increase in the expression of one or more neuronal and/or dopamine lineage markers at the protein level, Ascl1, Lmx1a, Nurr1, and FoxA2 mRNAs were delivered to iPSCs using squeeze processing, and then the expression of Asc11, Lmx1a, Nurr1, and FoxA2 was assessed as described below.

Briefly, as in the earlier Examples, iPSCs were treated with Accutase and dissociated into single cells. The iPSCs were prepared at a density of 1×10⁸ cells/mL, and a cocktail of codon-optimized mRNAs encoding Ascl1, Lmx1a, Nurr1, FoxA2, and PAC mRNAs was added to the cell suspension. The cells were squeeze processed at room temperature using a microfluidic constriction (length of 10 μm, width of 6 μm, and depth of 70 μm) at 60 psi. Cells squeeze processed without any cargo were used as controls.

Following squeeze processing, the cells that passed through the constriction were collected and transferred to StemFlex basal medium with supplement and incubated for 6 hours at 37° C. At 6 hours, the cells were washed with PBS to remove unwanted debris. A mixture of 1:1 ratio of StemFlex basal medium with supplement/N2B medium+B27 (100×) was then added. Total protein was collected from the cells at 4 and 8 hours for a Western blot analysis.

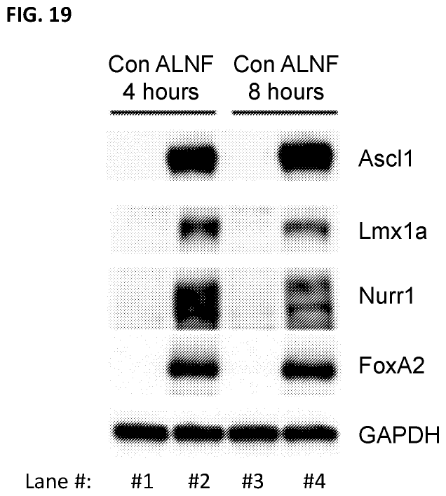

As show in FIG. 19, both at 4 hours and at 8 hours, the delivery of Ascl1, Lmx1a, Nurr1, and FoxA2 mRNA induced both neuronal and dopaminergic neuron lineage markers expression at the protein level. Collectively, the above results confirm that the squeeze processing methods described herein can deliver various reprogramming factors to cells (e.g., iPSCs and PBMCs), resulting in the successful differentiation of the cells into neurons.

### Example 14: Analysis of Neuronal and Dopamine Lineage Markers after Ascl1, Lmx1a, Nurr1, FoxA2, Pitx3, and EN1 mRNA Delivery Using Squeeze Processing

To further demonstrate the ability of squeeze processing methods provided herein to deliver mRNAs encoding certain reprogramming factors to cells and induce dopaminergic neuron differentiation, iPSCs were treated with Accutase and dissociated into single cells. The iPSCs were prepared at a density of 1×10⁸ cells/mL, and a cocktail of codon-optimized mRNAs encoding either (i) 6 TFs (Ascl1, Lmx1a, Nurr1, FoxA2, Pitx3, and EN1)+PAC or (ii) 4 TFs (Ascl1, Lmx1a, Nurr1, and FoxA2)+PAC was added to the cell suspension containing the prepared iPSCs. The cells (i.e., iPSCs) were squeeze processed at room temperature using a microfluidic constriction (length of 10 μm, width of 6 μm, and depth of 70 μm) at 60 psi. Cells squeeze processed without any cargo were used as controls.

Following squeeze processing, the cells that passed through the constriction were collected and transferred to StemFlex basal medium with supplement and incubated for 6 hours at 37° C. At 6 hours, the cells were washed with PBS to remove unwanted debris. A mixture of 1:1 ratio of StemFlex basal medium with N2B medium+B27 (100×) with 5 μg/ml puromycin was added. The mixture was incubated for 18 hours. The medium was then removed, and a mixture of NBM medium+B27 (50×)+BDNF+GDNF+CNTF was added at a concentration of 1:1000. Total RNA was collected from the cells at 8, 24, and 48 hours, and RT-qPCR was used to analyze the expression of general neuronal markers (NeuroD1) and dopamine lineage markers (FoxA2, Pitx3, Lmx1a, Nurr1, and TH).

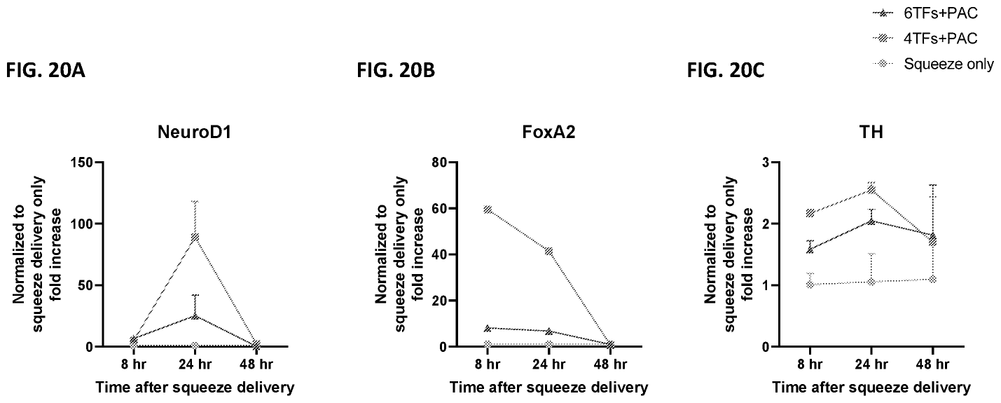

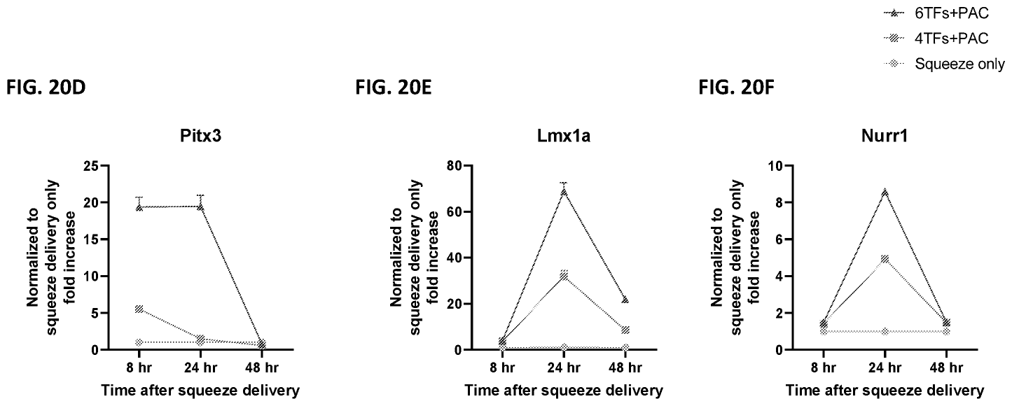

As show in FIGS. 20A-20F, the combined delivery of either of the mRNA cocktails, i.e., encoding 6 TFs (Ascl1, Nurr1, Lmx1a, FoxA2, Pitx3, and EN1) or encoding 4 TFs mRNAs (Ascl1, Nurr1, Lmx1a, and FoxA2), using squeeze processing induced both neuronal and dopaminergic neuron lineage marker expression in the iPSCs. The delivery of the mRNAs encoding the 6 TFs (Ascl1, FoxA2, Nurr1, Lmx1a, Pitx3, and EN1) using squeeze processing resulted in lower neuronal and floor plate progenitor marker (NeuroD1 and FoxA2—FIGS. 20A and 20B, respectively) expression but higher expression of the dopaminergic lineage specific markers (Pitx3, Lmx1a, and Nurr1—FIGS. 20D, 20E, and 20F, respectively), compared to cells that that received the mRNAs encoding the 4 TFs (Ascl1, FoxA2, Nurr1, and Lmx1a).

The above results further demonstrate that the squeeze processing methods described herein are capable of concurrently delivering multiple reprogramming factors (as many as six) to cells and thereby induce dopaminergic neuron differentiation.

### Example 15: Testing the Neuronal Differentiation Potential of CD34″ Human Hematopoietic Stem Cells (HSCs)

Next, the neurogenic potential of CD34+ hematopoietic stem cells (HSCs) as an autologous cell target to generate dopaminergic neurons was assessed. CD34+ cells are similar to PBMCs in their accessibility and translatability but are less differentiated, and therefore, it was proposed that they might be more permissive to transdifferentiate into a distant cell type.

Briefly, the transcription factor ASCL1 alone or the combination of transcription factors BRN2, ASCL1, MYT1L, NEUROG2 (collectively referred to herein as “BAMN”) was delivered to CD34+HSCs using squeeze processing. No contact (i.e., no squeeze processing) and squeeze processed with no transcription factors (empty squeeze) conditions were used as negative controls. After the squeeze delivery, cells were cultured in neuronal differentiation media (DMEM/F12, N2 (50×), B27 (100×), Pen/Strep (100×)) supplemented with 3 small molecules, i.e., forskolin (cAMP activator, 3 μM), dorsomorphin (BMP blocker, 2 μM), and SB431542 (TGF-B blocker, 10 μM). The cultured cells were harvested at 0 hr, 4 hr, 8 hr, and 24 hr after squeeze delivery, RNA extracted, and qPCR analysis was performed to measure the expression of genes involved in the downstream neuronal and dopaminergic gene activation, including Tuj1, ZBTB18, NeuroD1, NeuroD4, Th, and Nurr1. A subset of extracted RNA was used to check the delivery efficiency. Cell viability and recovery analysis (i.e., cell retention) was done after squeeze delivery and before the experiment endpoint.

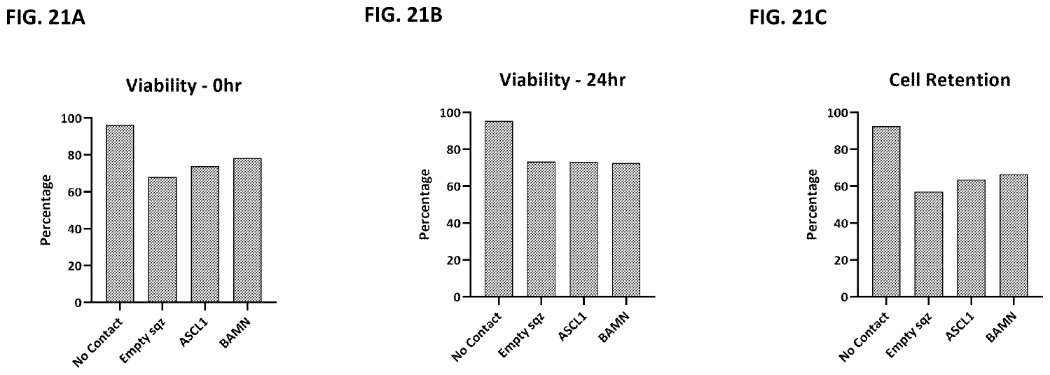

As shown in FIGS. 21A and 21B, significant percentage of the CD34+ cells remained viable (both at 0 hr and 24 hr after squeeze processing) under each of the conditions tested. The higher viability corresponded with higher post-squeeze recovery (FIG. 21C).

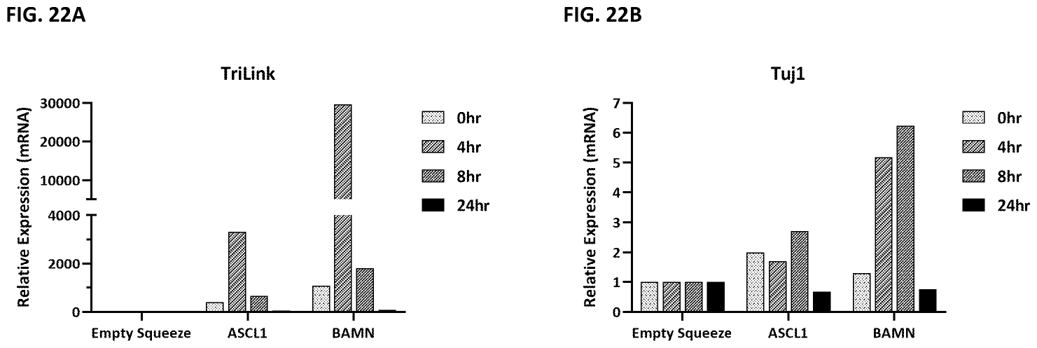

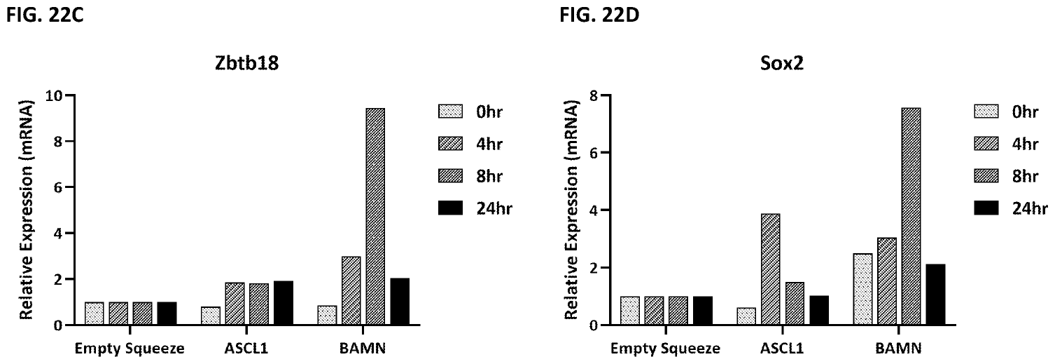

As to delivery efficiency, results of the qPCR analysis using TriLink universal primers confirmed that the reprogramming factors were successfully delivered to the cells using squeeze processing (FIG. 22A). The level of total delivered mRNA was proportionally higher in cells that received the BAMN mRNA cocktail compared to cells that received ASCL1 alone. Overall, these results further confirm the ability of the squeeze processing methods provided herein to efficiently deliver a payload into a cell.

As to the expression of the specific downstream neuronal and dopaminergic genes, results of the qPCR analysis showed the following: i) expression of the broad neuronal cell marker TUJ1 increased by about 2-3-fold after Ascl1 mRNA delivery, whereas the expression increased by about 5-7-fold upon BAMN mRNA delivery (FIG. 22B); ii) expression of the ASCL1 target proneural transcription factor ZBTB18 was increased by about 9-fold upon squeeze delivery of BAMN mRNA (FIG. 22C); and iii) expression of the neural stem cell marker SOX2 increased by about 4-fold after ASCL1 mRNA delivery alone, and increased by about ˜8-fold upon multiplex delivery of BAMN factors (FIG. 22D).

These data together show the broad neuronal differentiation potential of CD34+ human hematopoietic stem cells upon squeeze delivery of proneural reprogramming factors.

### Example 16: Efficiency and Specificity of Successive Squeeze Delivery in Human PBMCs

To achieve prolonged protein expression upon squeeze delivery of mRNAs, whether the recipient cells can be re-targeted or “hit” upon successive squeeze delivery across multiple days was assessed. Briefly, as further described below, different reporter mRNAs were used in each consecutive squeeze processing, and the proportion of single-, double-, and triple-reporter positive cells after each squeeze processing was assessed. Whether there were any specific PMBC subsets that were better targeted in the successive squeeze processing was also assessed.

More specifically, PBMCs (isolated as described in the earlier examples, see, e.g., Example 11) underwent three separate squeeze processing steps to deliver the following reporter mRNAs: (1) EGFP reporter mRNA in the first squeeze processing at t=0 hr, (2) mCherry reporter mRNA (RFP) in the second squeeze processing at t=24 hr, and (3) Blue Dextran reporter mRNA in the third squeeze processing at t=48 hr. Flow cytometry was performed 4 hours after each squeeze delivery, and the proportion of single-, double-, and triple-reporter positive PBMC subsets was assessed. No contact (i.e., no squeeze processing) condition was used as a negative control. Cells were cultured in neuronal differentiation media (DMEM/F12, N2 (50×), B27 (100×), Pen/Strep (100×)) to mimic the actual experimental conditions. Cell viability and recovery analysis were done before and after each squeeze delivery and before the experiment endpoint.

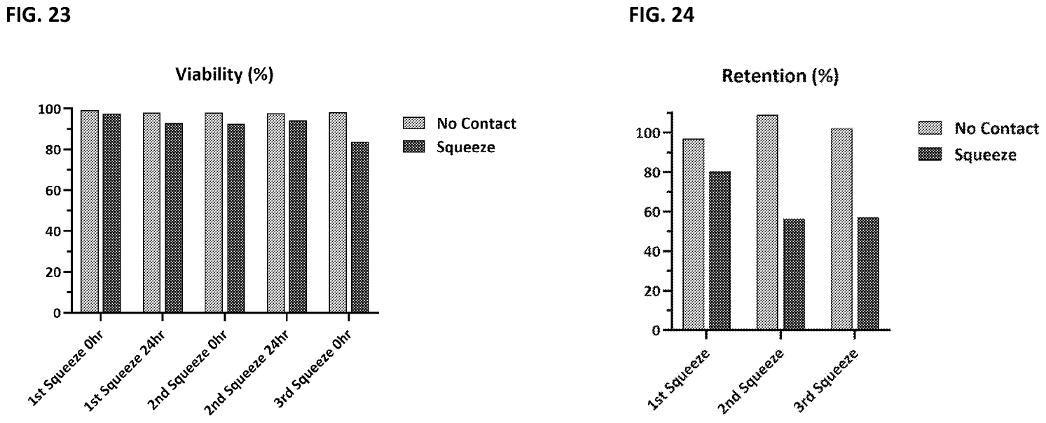

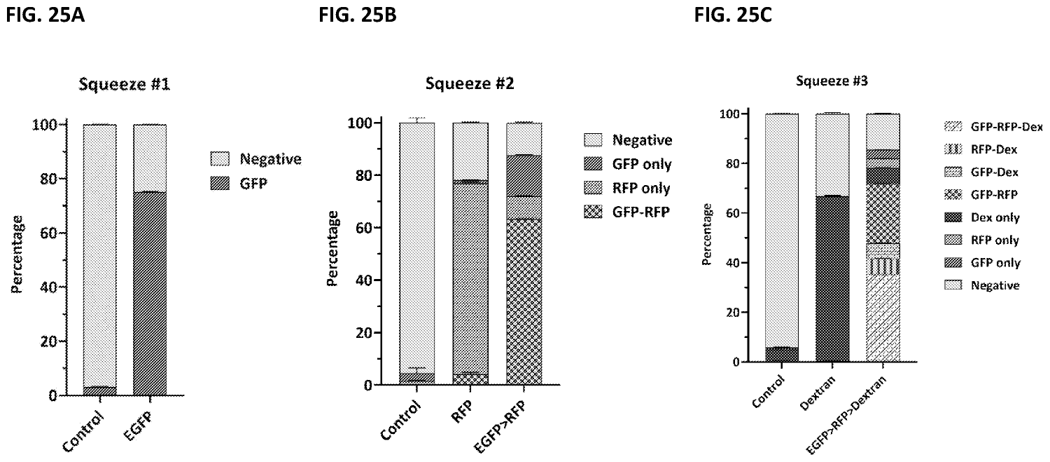

High cell viability (>80%) (FIG. 23) and cell retention (>50%) (FIG. 24) were observed at each of the three squeeze steps. Flow cytometry analysis of cells demonstrated that successive squeeze processes were highly efficient on targeting previously squeezed reporter positive cells. As shown, 71.4% of the cells were positive for GFP reporter after the first squeeze delivery (FIG. 25A). With the second squeeze delivery (RFP), ˜80% of previously squeezed GFP+ cells were targeted, resulting in 63.4% of the cells being double positive for GFP and RFP (FIG. 25B). Similarly, with the third squeeze delivery (Blue Dextran), ˜80% of double squeeze delivered GFP and RFP positive cells were targeted, resulting in ˜35% of the total cell population being triple positive for GFP, RFP, and Dextran (FIG. 25C).

These results suggests that prolonged protein expression of reprogramming factors is feasible via multiple doses of squeeze delivery of mRNAs.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

What is claimed is:

1. A method of producing a neuron, comprising:
passing a cell suspension, which comprises a population of cells, through a constriction under one or more parameters,
wherein passing the cell suspension through the constriction under the one or more parameters deforms one or more cells of the population of cells, and thereby, causing a perturbation in the cell membrane of the one or more cells, and
wherein the perturbation allows a reprogramming factor to enter the one or more cells, and thereby, reprograms the one or more cells into a neuron.

2. The method of claim 1, comprising contacting the population of cells with the reprogramming factor prior to passing of the cell suspension through the constriction.

3. The method of claim 1 or 2, comprising contacting the population of cells with the reprogramming factor during the passing of the cell suspension through the constriction.

4. The method of claim 3, wherein the population of cells are contacted with the reprogramming factor during the passing of the cell suspension through the constriction.

5. The method of any one of claims 1 to 3, comprising contacting the population of cells with the reprogramming factor after the passing of the cell suspension through the constriction.

6. The method of claim 5, wherein the population of cells are contacted with the reprogramming factor after the passing of the cell suspension through the constriction.

7. The method of any one of claims 1 to 6, wherein the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

8. The method of claim 7, wherein the nucleic acid comprises a DNA, RNA, or both.

9. The method of claim 8, wherein the DNA comprises a recombinant DNA, a cDNA, a genomic DNA, or combinations thereof.

10. The method of claim 8, wherein the RNA comprises a siRNA, a mRNA, a microRNA (miRNA), a lncRNA, a tRNA, a shRNA, a self-amplifying mRNA (saRNA), or combinations thereof.

11. The method of claim 10, wherein the RNA is a mRNA.

12. The method of claim 10, wherein the RNA is a siRNA.

13. The method of claim 10, wherein the RNA is a shRNA.

14. The method of claim 7, wherein the small molecule comprises an impermeable small molecule.

15. The method of any one of claims 1 to 14, wherein the reprogramming factor comprises a transcription factor, wherein the transcription factor is capable of reprogramming the one or more cells into a neuron.

16. The method of claim 15, wherein the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor related 1 protein (Nurr1), LIM Homeobox Transcription Factor 1 Alpha (Lmx1a), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124 (mir124), or combinations thereof.

17. The method of any one of claims 1 to 16, wherein the reprogramming factor inhibits the expression and/or activity of p53.

18. The method of claim 17, wherein the reprogramming factor comprises a shRNA targeting p53.

19. The method of claim 10, wherein the reprogramming factor comprises a miRNA.

20. The method of claim 19, wherein the miRNA comprises miR-124.

21. A method of inducing the reprogramming of a cell comprising:
passing a cell suspension, which comprises a population of cells, through a constriction under one or more parameters,
wherein passing the cell suspension through the constriction under the one or more parameters deforms one or more cells of the population of cells, and thereby, causing a perturbation in the cell membrane of the one or more cells, and
wherein the perturbation allows a reprogramming factor to enter the one or more cells, and thereby, induce the reprogramming of the one or more cells.

22. The method of claim 21, comprising contacting the population of cells with the reprogramming factor prior to passing of the cell suspension through the constriction.

23. The method of claim 21 or 22, comprising contacting the population of cells with the reprogramming factor during the passing of the cell suspension through the constriction.

24. The method of claim 23, wherein the population of cells are contacted with the reprogramming factor during the passing of the cell suspension through the constriction.

25. The method of any one of claims 21 to 24, comprising contacting the population of cells with the reprogramming factor after the passing of the cell suspension through the constriction.

26. The method of claim 25, wherein the population of cells are contacted with the reprogramming factor after the passing of the cell suspension through the constriction.

27. The method of any one of claims 21 to 26, wherein the reprogramming factor is capable of reprogramming the one or more cells to a neuron.

28. The method of any one of claims 21 to 27, wherein the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

29. The method of claim 28, wherein the nucleic acid comprises a DNA, RNA, or both.

30. The method of claim 29, wherein the DNA comprises a recombinant DNA, a cDNA, a genomic DNA, or combinations thereof.

31. The method of claim 29, wherein the RNA comprises a siRNA, a mRNA, a microRNA (miRNA), a lncRNA, a tRNA, a shRNA, a self-amplifying mRNA (saRNA), or combinations thereof.

32. The method of claim 31, wherein the RNA is a mRNA.

33. The method of claim 31, wherein the RNA is a siRNA.

34. The method of claim 31, wherein the RNA is a shRNA.

35. The method of claim 28, wherein the small molecule comprises an impermeable small molecule.

36. The method of any one of claims 21 to 35, wherein the reprogramming factor comprises a transcription factor, wherein the transcription factor is capable of reprogramming the one or more cells into a neuron.

37. The method of claim 36, wherein the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor related 1 protein (Nurr1), LIM Homeobox Transcription Factor 1 Alpha (Lmx1a), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124 (mir124), or combinations thereof.

38. The method of any one of claims 21 to 37, wherein the reprogramming factor inhibits the expression and/or activity of p53.

39. The method of claim 38, wherein the reprogramming factor comprises a shRNA targeting p53.

40. The method of claim 31, wherein the reprogramming factor comprises a miRNA.

41. The method of claim 40, wherein the miRNA comprises miR-124.

42. The method of any one of claims 21 to 41, wherein the one or more cells are differentiated into a neuron.

43. A method of increasing the delivery of a reprogramming factor into a cell, comprising modulating one or more parameters under which a cell suspension, comprising a population of cells, is passed through a constriction, wherein the population of cells are in contact with the reprogramming factor, and wherein the one or more parameters increase the delivery of the reprogramming factor into one or more cells of the population of cells compared to a reference parameter.

44. The method of claim 43, comprising contacting the population of cells with the reprogramming factor prior to passing of the cell suspension through the constriction.

45. The method of claim 43 or 44, comprising contacting the population of cells with the reprogramming factor during the passing of the cell suspension through the constriction.

46. The method of claim 45, wherein the population of cells are contacted with the reprogramming factor during the passing of the cell suspension through the constriction.

47. The method of any one of claims 43 to 46, comprising contacting the population of cells with the reprogramming factor after the passing of the cell suspension through the constriction.

48. The method of claim 47, wherein the population of cells are contacted with the reprogramming factor after the passing of the cell suspension through the constriction.

49. The method of any one of claims 43 to 48, wherein the delivery of the reprogramming factor into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the reprogramming factor into a corresponding cell using the reference parameter.

50. The method of any one of claims 1 to 49, wherein the one or more cells of the population of cells comprises stem cells, somatic cells, or both.

51. The method of claim 50, wherein the stem cells are induced pluripotent stem cells (iPSCs), embryonic stem cells, tissue-specific stem cells, mesenchymal stem cells, or combinations thereof.

52. The method of claim 51, wherein the stem cells are iPSCs.

53. The method of claim 50, wherein the somatic cells comprise blood cells.

54. The method of claim 53, wherein the blood cells are PBMCs.

55. The method of claim 54, wherein the PBMCs comprise immune cells.

56. The method of claim 55, wherein the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

57. The method of any one of claims 1 to 56, wherein the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or combinations thereof.

58. The method of claim 57, wherein the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

59. The method of any one of claim 57 or 58, wherein the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

60. The method of any one of claims 1 to 59, wherein the constriction is contained within a microfluidic chip.

61. The method of any one of 1 to 60, wherein the diameter of the constriction is about 20%, about 30%, about 40%, about 50%, about 60%, about 70%, about 80%, about 90%, or about 99% of the diameter of the one or more cells of the population of cells.

62. The method of any one of claims 1 to 61, wherein the length of the constriction is up to 100 μm.

63. The method of claim 62, wherein the length of the constriction is less than 1 μm.

64. The method of claim 62, wherein the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm.

65. The method of claim 62, wherein the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm.

66. The method of any one of claims 1 to 65, wherein the width of the constriction is up to about 10 μm.

67. The method of claim 66, wherein the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

68. The method of claim 66, wherein the width of the constriction is between about 3 μm to about 10 μm.

69. The method of claim 68, wherein the width of the constriction is about 3 μm, about 4 μm, about 5 μm, about 6 μm, about 7 μm, about 8 μm, about 9 μm, or about 10 μm.

70. The method of any one of claims 1 to 69, wherein the depth of the constriction is at least about 1 μm.

71. The method of claim 70, wherein the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

72. The method of claim 70 or 71, wherein the depth of the constriction is about 5 μm to about 90 μm.

73. The method of claim 72, wherein the depth is about 5 μm, about 10 μm, about 20 μm, about 30 μm, about 40 μm, about 50 μm, about 60 μm, about 70 μm, about 80 μm, or about 90 μm.

74. The method of any one of claims 1 to 73, wherein the population of cells are contacted with multiple reprogramming factors, such that at least two or more of the multiple reprogramming factors are cable of entering the one or more cells after the perturbation of the cell membrane.

75. The method of claim 74, wherein multiple reprogramming factors comprise at least about 2, at least about 3, at least about 4, at least about 5, at least about 6, at least about 7, at least about 8, at least about 9, or at least about 10 or more reprogramming factors.

76. The method of claim 75, wherein the multiple reprogramming factors are delivered into the cells concurrently.

77. The method of claim 75, wherein one or more of the reprogramming factors are delivered into the cells sequentially.

78. The method of any one of claims 1 to 77, comprising passing the cell suspension through a plurality of constrictions.

79. The method of claim 78, wherein the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions.

80. The method of claim 78 or 79, wherein each constriction of the plurality of constrictions are the same.

81. The method of claim 78 or 79, wherein one or more of the constrictions of the plurality of constrictions are different.

82. The method of claim 81, wherein the one or more of the constrictions differ in their length, depth, width, or combinations thereof.

83. The method of any one of claims 78 to 82, wherein each constriction of the plurality of constrictions is associated with the same reprogramming factor.

84. The method of any one of claims 78 to 82, wherein one or more of the plurality of constrictions is associated with a different reprogramming factor.

85. The method of claim 84, wherein the plurality of constrictions comprise a first constriction associated with a first reprogramming factor and a second constriction associated with a second reprogramming factor, wherein the cell suspension passes through the first constriction such that the first reprogramming factor is delivered to one or more cells of the plurality of cells, and then the cell suspension passes through the second constriction such that the second reprogramming factor is delivered to the one or more cells of the plurality of cells.

86. The method of claim 85, wherein the cell suspension is passed through the second constriction at least about 1 minute, at least about 30 minutes, at least about 1 hour, at least about 6 hours, at least about 12 hours, or at least about 1 day after the cell suspension is passed through the first constriction.

87. The method of any one of claims 1 to 86, further comprising contacting the plurality of cells with an additional compound.

88. The method of claim 87, wherein the additional compound comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

89. The method of claim 87, wherein the additional compound is a nucleic acid encoding an enzyme that confers resistance to an antibiotic.

90. The method of any one of claims 87 to 89, wherein the contacting of the additional compound with the plurality of cells occurs concurrently with the reprogramming factor.

91. The method of any one of claims 87 to 89, wherein the contacting of the additional compound with the plurality of cells occurs prior to or after the contacting of the plurality of cells with the reprogramming factor.

92. The method of any one of claims 89 to 91, further comprising collecting the cell suspension that passed through the constriction and treating the cell suspension with the antibiotic.

93. The method of any one of claims 89 to 92, wherein the enzyme comprises puromycin-N-acetyltransferase and the antibiotic is puromycin.

94. The method of claim 92 or 93, wherein after the treatment with the antibiotic, the proportion of neurons present in the cell suspension is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold.

95. A composition comprising a population of differentiated cells produced by the methods of any one of claims 1 to 94.

96. A composition comprising a population of cells and a reprogramming factor under one or more parameters resulting in deformation of one or more cells of the population of cells, wherein the one or more cells comprise a perturbation in the cell membrane sufficient to allow the reprogramming factor to enter the one or more cells.

97. The composition of claim 96, wherein the one or more cells of the population of cells comprises stem cells, somatic cells, or both.

98. The composition of claim 97, wherein the stem cells are induced pluripotent stem cells (iPSCs), embryonic stem cells, tissue-specific stem cells, mesenchymal stem cells, or combinations thereof.

99. The composition of claim 98, wherein the stem cell is iPSC.

100. The composition of claim 97, wherein the somatic cells comprise blood cells.

101. The composition of claim 100, wherein the blood cells are PBMCs.

102. The composition of claim 101, wherein the PBMCs comprise immune cells.

103. The composition of claim 102, wherein the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

104. The composition of any one of claims 96 to 103, wherein the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

105. The composition of claim 104, wherein the nucleic acid comprises a DNA, RNA, or both.

106. The composition of claim 105, wherein the DNA comprises a recombinant DNA, a cDNA, a genomic DNA, or combinations thereof.

107. The composition of claim 105, wherein the RNA comprises a siRNA, a mRNA, a miRNA, a lncRNA, a tRNA, a shRNA, a self-amplifying mRNA (saRNA), or combinations thereof.

108. The composition of claim 107, wherein the RNA is a mRNA.

109. The composition of any one of claims 96 to 108, wherein the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a payload concentration; a viscosity, osmolarity, salt concentration, serum content, and/or pH of the cell suspension; time in the constriction; shear rate in the constriction; type of payload or combinations thereof.

110. The composition of claim 109, wherein the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

111. The composition of any one of claim 109 or 110, wherein the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

112. The composition of any one of claims 96 to 111, wherein the constriction is contained within a microfluidic chip.

113. The composition of any one of claims 96 to 112, wherein the length of the constriction is up to 100 μm.

114. The composition of claim 113, wherein the length of the constriction is less than 1 μm.

115. The composition of claim 113, wherein the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm.

116. The composition of claim 113, wherein the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm.

117. The composition of any one of claims 96 to 116, wherein the width of the constriction is up to about 10 μm.

118. The composition of claim 117, wherein the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

119. The composition of claim 117, wherein the width of the constriction is between about 3 μm to about 10 μm.

120. The composition of claim 119, wherein the width of the constriction is about 3 μm, about 4 μm, about 5 μm, about 6 μm, about 7 μm, about 8 μm, about 9 μm, or about 10 μm.

121. The composition of any one of claims 96 to 120, wherein the depth of the constriction is at least about 1 μm.

122. The composition of claim 121, wherein the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

123. The composition of claim 121 or 122, wherein the depth of the constriction is about 5 μm to about 90 μm.

124. The composition of claim 123, wherein the depth is about 5 μm, 10 μm, about 20 μm, about 30 μm, about 40 μm, about 50 μm, about 60 μm, about 70 μm, about 80 μm, or about 90 μm.

125. A cell comprising a perturbation in the cell membrane due to one or more parameters which deform the cell thereby causing the perturbation in the cell membrane of the cell such that a reprogramming factor can enter the cell.

126. A cell comprising a reprogramming factor, wherein the reprogramming factor entered the cell through a perturbation in the cell membrane due to one or more parameters which deformed the cell thereby causing the perturbation in the cell membrane of the cell such that the reprogramming factor entered the cell.

127. The cell of claim 125 or 126, wherein the cell comprises stem cells, somatic cells, or both.

128. The cell of claim 127, wherein the stem cells are induced pluripotent stem cells (iPSCs), embryonic stem cells, tissue-specific stem cells, mesenchymal stem cells, or combinations thereof.

129. The cell of claim 128, wherein the stem cell is iPSC.

130. The cell of claim 127, wherein the somatic cells comprise blood cells.

131. The cell of claim 130, wherein the blood cells are PBMCs.

132. The cell of claim 131, wherein the PBMCs comprise immune cells.

133. The cell of claim 132, wherein the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

134. The cell of any one of claims 125 to 133, wherein the reprogramming factor comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof.

135. The cell of claim 134, wherein the nucleic acid comprises a DNA, RNA, or both.

136. The cell of claim 135, wherein the DNA comprises a recombinant DNA, a cDNA, a genomic DNA, or combinations thereof.

137. The cell of claim 135, wherein the RNA comprises a siRNA, a mRNA, a miRNA, a lncRNA, a tRNA, a shRNA, a self-amplifying mRNA (saRNA), or combinations thereof.

138. The cell of claim 137, wherein the RNA is a mRNA.

139. The cell of claim 137, wherein the RNA is siRNA.

140. The cell of claim 137, wherein the RNA is shRNA.

141. The cell of claim 134, wherein the small molecule comprises an impermeable small molecule.

142. The cell of any one of claims 125 to 141, wherein the one or more parameters are selected from a cell density; a pressure; a length, width, and/or depth of the constriction; a diameter of the constriction; a diameter of the cells; a temperature; an entrance angle of the constriction; an exit angle of the constriction; a length, width, and/or width of an approach region; a surface property of the constriction (e.g., roughness, chemical modification, hydrophilic, hydrophobic); an operating flow speed; a payload concentration; a viscosity, osmolarity, salt concentration, serum content, and/or pH of the cell suspension; time in the constriction; shear rate in the constriction;
type of payload or combinations thereof.

143. The cell of claim 142, wherein the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

144. The cell of claim 142 or 143, wherein the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

145. The cell of any one of claims 125 to 144, wherein the constriction is within a microfluidic chip.

146. The cell of any one of claims 125 to 145, wherein the diameter of the constriction is about 20%, about 30%, about 40%, about 50%, about 60%, about 70%, about 80%, about 90%, or about 99% of the diameter of the one or more cells of the population of cells.

147. The cell of any one of claims 125 to 146, wherein the length of the constriction is up to 100 μm.

148. The cell of claim 147, wherein the length of the constriction is less than 1 μm.

149. The cell of claim 147, wherein the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm.

150. The cell of claim 147, wherein the length of the constriction is about 1 μm, about 5 μm, about 10 μm, about 20 μm, about 30 μm, about 40 μm, about 50 μm, about 60 μm, about 70 μm, about 80 μm, about 90 μm, or about 100 μm.

151. The cell of any one of claims 125 to 150, wherein the width of the constriction is up to about 10 μm.

152. The cell of claim 151, wherein the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm.

153. The cell of claim 151, wherein the width of the constriction is between about 3 μm to about 10 μm.

154. The cell of claim 153, wherein the width of the constriction is about 3 μm, 4 μm, about 5 μm, about 6 μm, about 7 μm, about 8 μm, about 9 μm, or about 10 μm.

155. The cell of any one of claims 125 to 154, wherein the depth of the constriction is at least about 1 μm.

156. The cell of claim 155, wherein the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

157. The cell of claim 155 or 156, wherein the depth of the constriction is about 5 μm to about 90 μm.

158. The cell of claim 157, wherein the depth is about 5 μm, 10 μm, about 20 μm, about 30 μm, about 40 μm, about 50 μm, about 60 μm, about 70 μm, about 80 μm, or about 90 μm.

159. A method of treating a neurological disorder in a subject in need thereof, comprising administering to the subject a plurality of neurons, wherein the neurons are produced by the methods of any one of claims 1 to 94.

160. A method of treating a neurological disorder in a subject in need thereof, comprising administering to the subject the composition of any one of claims 95 to 124 or the cell of any one of claims 126 to 158.

161. The method of claim 152 or 153, wherein the neurological disorder comprises a Parkinson's disease.
